(19) United States
(12) Patent Application Publication
(10) Pub. No.: US 2024/0060072 A1
(43) Pub. Date: Feb. 22, 2024

(54) METHOD AND COMPOSITION FOR TREATING NON-ALCOHOLIC FATTY LIVER DISEASE, ASSOCIATED CONDITIONS AND SYMPTOMS

(71) Applicant: THE HONG KONG POLYTECHNIC UNIVERSITY, Hong Kong (CN)

(72) Inventors: Chi Ming WONG, Hong Kong (CN); Mengyao WU, Hong Kong (CN)

(21) Appl. No.: 18/447,362

(22) Filed: Aug. 10, 2023

Related U.S. Application Data
(60) Provisional application No. 63/370,948, filed on Aug. 10, 2022.

(51) Int. Cl. C12N 15/113; C12N 15/86; A61P 1/16

(52) U.S. Cl. CPC C12N 15/1138; C12N 15/86; A61P 1/16; C12N 2310/11; C12N 2750/14141; C12N 2710/10141

(57) ABSTRACT

The present invention provides a method for treating non-alcoholic fatty liver disease (NAFLD), associated conditions and symptoms. In particular, the method includes administering to a subject suffering from or susceptible to NAFLD a composition including therapeutic nucleic acids to reduce G protein-coupled receptor 110 (GPR110) expression in liver. The present invention also provides a pharmaceutical composition for treating NALFD or alleviating conditions and symptoms associated therewith.

### CROSS-REFERENCE TO RELATED APPLICATION

This application claims priority from the U.S. provisional patent application Ser. No. 63/370,948 filed Aug. 10, 2022, the disclosure of which is incorporated herein by reference in its entirety.

### REFERENCE TO SEQUENCE DISCLOSURE

A sequence listing file named “P24582US01_sequence_listing.xml” in ST.26 format with a file size of 42 kb created on Aug. 9, 2023 is incorporated herein by reference.

### TECHNICAL FIELD

The present invention relates to a method for treating non-alcoholic fatty liver disease (NAFLD), conditions and symptoms associated therewith. In particular, the method includes using RNA interference (RNAi) techniques to reduce hepatic G protein-coupled receptor 110 (GPR110) expression in normal, NAFLD-susceptible and NAFLD subjects.

### BACKGROUND

The following references are cited and discussed hereinafter:
1. Falvella F S, Pascale R M, Gariboldi M, Manenti G, De Miglio M R, Simile M M, et al. Stearoyl-CoA-desaturase 1 (Scd1) gene overexpression is associated with genetic predisposition to hepatocarcinogenesis in mice and rats. Carcinogenesis 2002; 23: 1933-1936.
2. Fredriksson R, Lagerstrom M C, Hoglund P J, Schioth H B. Novel human G protein-coupled receptors with long N-terminals containing GPS domains and Ser/Thr-rich regions. FEBS letters 2002; 531: 407-414.
3. Kurtz R, Anderman M F, Shepard B D. GPCRs get fatty: the role of G protein-coupled receptor signaling in the development and progression of nonalcoholic fatty liver disease. American Journal of Physiology Gastrointestinal and Liver Physiology 2021; 320: G304-G318.
4. Ma B, Zhu J, Tan J, Mao Y, Tang L, Shen C, et al. Gpr110 deficiency decelerates carcinogen-induced hepatocarcinogenesis via activation of the IL-6/STAT3 pathway. Am J Cancer Res 2017;7:433-447.
5. Oballa R M, Belair L, Black W C, Bleasby K, Chan C C, Desroches C, et al. Development of a liver-targeted stearoyl-CoA desaturase (SCD) inhibitor (MK-8245) to establish a therapeutic window for the treatment of diabetes and dyslipidemia. Journal of medicinal chemistry 2011;54:5082-5096.

Liver is a vital organ as it is the site for undergoing a number of crucial physiological processes including digestion, metabolism, immunity and storage of nutrients. Over-storage of lipid in the hepatocytes not caused by alcohol is known as non-alcoholic fatty liver disease (NAFLD), which is the most common liver pathological condition. The development of NAFLD is contributed by many factors such as lipid metabolism disorders, over- or mal-nutrition, inflammation, virus infection, or liver injuries. NAFLD usually does not entail any symptoms at early stages. However, if left untreated, NAFLD accounts for approximately 85% of all chronic noncommunicable diseases (NCDs), such as type 2 diabetes mellitus (T2DM), cardiovascular disease (CVD) and chronic kidney disease (CKD). In addition, NAFLD may progress to non-alcoholic steatohepatitis (NASH) with fibrosis, cirrhosis or even hepatocellular carcinoma (HCC).

Improvement in managing NAFLD helps resolve at least partially the progression of the above diseases. Stopping the progression of NAFLD by lifestyle modifications such as increasing physical exercise activity and reduction of hypercaloric diet are only effective during the early stages before there is fibrosis. G protein-coupled receptors (GPCRs) are the largest and most diverse family of membrane receptor that play important roles in regulating most cellular and physiological processes. A few GPCRs have been shown to play key roles in NAFLD and modulating their activities to ameliorate liver-related metabolic syndrome was proposed as NAFLD treatment (Kurtz et al., 2021; Fredriksson et al., 2002). However, currently proposed targets for GPCR-medicated NAFLD treatment are not exclusively expressed in hepatocytes, thus the potential side effects on other organs have not been fully considered.

G protein-coupled receptor 110 (GPR110), an oncogene, or called ADGRF1, is an orphan receptor that belongs to Family VI adhesion-GPCRs (aGPCRs) together with ADGRF2-5 (formerly GPR111, GPR113, GPR115 and GPR116 respectively). The N terminus of ADGRF1 contains a GPCR proteolysis site (GPS) and a SEA (Sperm protein, Enterokinase and Agrin) domain. GPR110 (or ADGRF1) is a receptor for N-docosahexaenoylethanolamine (synaptamide), an endogenous metabolite of docosahexaenoic acid, that potently promotes neurogenesis, neuritogenesis and synaptogenesis. ADGRF1 knockout mice showed significant deficits in object recognition and spatial memory. GPR110 has also been shown high expression in numerous cancer types and involves in cell survival, migration, invasion and proliferation. Ma et al. (2017) reported that deficiency GPR110 can decelerate carcinogen-induced hepatocarcinogenesis in adult mice. GPR110 has been found to be mostly expressed in the liver of healthy individuals, but the expression of hepatic GPR110 was dramatically decreased in obese subjects. These interesting findings provide some insights into the development of a targeted therapy in treating NAFLD and conditions or symptoms associated therewith.

### SUMMARY OF INVENTION

Accordingly, a first aspect of the present invention provides a method for treating non-alcoholic fatty liver disease (NAFLD), conditions and symptoms associated therewith. The method includes using RNA interference (RNAi) techniques to reduce G protein-coupled receptor 110 (GPR110) expression in a subject. Various in vitro and in vivo data provided herein suggest that GPR110 regulates hepatic lipid metabolism through controlling a downstream target of GPR110, stearoyl-coA desaturase 1 (SCD1), which is a crucial enzyme in hepatic de novo lipogenesis. High expression level of SCD1 is known to be genetically susceptible to hepatocarcinogenesis (Falvella et al., 2002). The data also suggest that down-regulation of GPR110 expression can potentially serve as a protective mechanism to stop the over-accumulation of fat in the liver in obese subjects. Improvements of hepatic steatosis in an in vivo NAFLD disease model and lipid profile of hepatocytes via treatment with liver-specific SCD1 inhibitor and specific shRNAs against SCD1 in primary hepatocytes, respectively, also support the role of GPR110 expression in regulation of NAFLD progression.

Therefore, in the first aspect, the method for treating (or alleviating the progression of) NAFLD and conditions associated therewith includes administering to a subject a composition comprising therapeutic nucleic acids to reduce GPR110 expression in liver of the subject.

In certain embodiments, the composition is administered via intravenous injection, subcutaneous injection, or oral administration.

In certain embodiments, the therapeutic nucleic acids are capable of gene silencing of GPR110.

In certain embodiments, the therapeutic nucleic acids include small interfering RNA (siRNA), short-hairpin RNA (shRNA), micro RNA (miRNA), RNA induced silencing complex (RICS), or a complex of clustered regularly interspaced short palindromic repeats (CRISPR) with CRISPR-associated protein 9 (Cas9).

In certain embodiments, the therapeutic nucleic acids are hepatic GPR110-specific antisense oligonucleotides (ASOs).

In certain embodiments, the hepatic GPR110-specific ASOs include two individual ASOs with different RNA sequences specific to decrease the expression level of GPR110 messenger RNA (mRNA) in the liver of the subject.

In certain embodiments, the therapeutic nucleic acids are siRNAs each having a nucleotide sequence selected from one of SEQ ID NOs: 35-38.

In certain embodiments, the composition also includes any inhibitor or antagonist for treating various cancers such as liver cancer, glioma and kidney cancer at which an elevated GPR110 expression level is found.

In certain embodiments, the composition further includes a viral vector.

In certain embodiments, the viral vector includes adenoviral, adeno-associated viral, retroviral, or lentiviral vector.

In certain embodiments, the conditions or symptoms associated with the NAFLD include hepatic steatosis, and chronic noncommunicable diseases (NCDs) such as type 2 diabetes mellitus (T2DM), cardiovascular disease (CVD), hypertriglyceridemia, atherosclerosis, and chronic kidney disease (CKD), non-alcoholic steatohepatitis (NASH) with fibrosis, cirrhosis and hepatocellular carcinoma (HCC).

In certain embodiments, the subject includes non-human animals and humans.

A second aspect of the present invention provides a pharmaceutical composition comprising therapeutic nucleic acids to reduce G protein-coupled receptor 110 (GPR110) expression in liver of a subject for treating non-alcoholic fatty liver disease (NAFLD), conditions and symptoms associated therewith.

In certain embodiments, the therapeutic nucleic acids are capable of gene silencing of GPR110.

In certain embodiments, the therapeutic nucleic acids include small interfering RNA (siRNA), short-hairpin RNA (shRNA), micro RNA (miRNA), RNA induced silencing complex (RICS), or a complex of clustered regularly interspaced short palindromic repeats (CRISPR) with CRISPR-associated protein 9 (Cas9).

In certain embodiments, the therapeutic nucleic acids are hepatic GPR110-specific antisense oligonucleotides (ASOs).

In certain embodiments, the hepatic GPR110-specific ASOs include two individual ASOs with different RNA sequences specific to decrease the expression level of GPR110 mRNA in the liver of the subject.

In certain embodiments, the therapeutic nucleic acids are siRNAs each having a nucleotide sequence selected from one of SEQ ID NOs: 35-38.

In certain embodiments, the pharmaceutical composition is administered via intravenous injection, subcutaneous injection, or oral administration.

In certain embodiments, the conditions or symptoms associated with the NAFLD include hepatic steatosis, and chronic noncommunicable diseases (NCDs) such as type 2 diabetes mellitus (T2DM), cardiovascular disease (CVD), hypertriglyceridemia, atherosclerosis, and chronic kidney disease (CKD), non-alcoholic steatohepatitis (NASH) with fibrosis, cirrhosis and hepatocellular carcinoma (HCC).

In certain embodiments, the subject includes non-human animals and humans.

In certain embodiments, the composition also includes any inhibitor or antagonist for treating various cancers such as liver cancer, glioma and kidney cancer at which an elevated GPR110 expression level is found.

In certain embodiments, the pharmaceutical composition further includes a viral vector.

In certain embodiments, the viral vector includes adenoviral, adeno-associated viral, retroviral, or lentiviral vector.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIGS. 1A-1G show that G-protein coupled receptor 110 (GPR110) is mainly expressed in the liver and its expression is downregulated after feeding standard chow diet (STC) or high-fat diet (HFD) to a mouse model, in which data represents as mean±SEM; repeated with three independent experiments; P value analyzed by two-tailed Student's t test. *P<0.05, **P<0.01, ***P<0.001:

FIG. 1A shows mRNA expression levels of GPR110 in different organs as determined by RT-qPCR analysis (n=5);

FIG. 1B shows representative immunoblotting analyses of GPR110 expression in different tissues of the mouse model after feeding STC for 8 weeks (n=3);

FIG. 1C shows mRNA expression levels of GPR110, CD11b and albumin in factions of hepatocyte or NPC isolated from STC-fed mouse livers as determined by RT-qPCR;

FIG. 1D shows representative immunoblotting analyses of GPR110, CD11b and albumin in fractions of hepatocytes or non-parenchymal cells (NPCs) isolated from the mouse livers fed with STC (left panel), where each lane is a sample from different individual; and quantification of protein expression levels of GPR110, CD11b and albumin (right panel), where protein expression levels were normalized to the expression of 13-actin, and the fraction of hepatocytes was set as 1 for fold-change calculation;

FIG. 1E shows mRNA expression levels of FGF21 in mice liver fed with 0, 2, 4, 6, 8 weeks of HFD as determined by RT-qPCR;

FIG. 1F shows mRNA expression levels of GPR110 in mice liver fed with 0, 2, 4, 6, 8 weeks of HFD as determined by RT-qPCR;

FIG. 1G shows representative immunoblotting analyses of GPR110 in mice fed with either STC or HFD for 8 weeks (left panel); and quantification of protein expression levels of GPR110 (right panel), where protein expression levels were normalized to the expression of 13-tubulin; the sample from STC mice were set as 1 for fold-change calculation; and each lane is a sample from different individual.

FIGS. 2A-2L show an overexpression of GPR110 in hepatocytes exaggerates metabolic dysregulation by HFD treatment to a mouse model, in which data represents as mean±SEM; repeated with three independent experiments; P value analyzed by two-tailed Student's t test. *P<0.05, **P<0.01, ***P<0.001; n=8 per group:

FIG. 2A schematically depicts a scheme of viral treatments to the mouse model according to certain embodiments, where 8-week-old male C57BL/6J mice were infected with 3×10¹¹ copies of rAAV encoding GPR110 (rAAV-GPR110) via intravenous injection (i.v.) or control (rAAV-GFP) via i.v. after feeding HFD for 8 weeks;

FIG. 2B shows hepatic mRNA expression levels of GPR110 from rAAV-GPR110 mouse livers in fractions of hepatocytes or NPCs isolated from HFD-fed mice livers as determined by RT-qPCR, where mRNA expression levels of the target genes were normalized to the expression of mouse GAPDH;

FIG. 2C shows immunoblotting analyses of GPR110, CD11b and albumin from rAAV-GPR110 mouse livers in factions of hepatocytes or NPCs isolated from HFD-fed mouse livers, where each lane is a sample from a different individual, and negative control (rAAV-NC) group was set as 1 for fold-change calculation;

FIG. 2D shows the change of body weight of rAAV-GPR110 mice and control biweekly upon i.v. injection;

FIG. 2E shows the percentage change of fat mass in rAAV-GPR110 mice and control between week 0 and week 12;

FIG. 2F shows the percentage change of lean mass in rAAV-GPR110 mice and control between week 0 and week 12;

FIG. 2G shows fasting blood glucose levels in rAAV-GPR110 mice and control measured biweekly upon i.v. injection;

FIG. 2H shows the change of fasting serum insulin levels in rAAV-GPR110 mice and control between week 0 and week 12;

FIG. 2I shows the change of homeostasis model assessment-estimated insulin resistance (HOMA-IR) index in rAAV-GPR110 mice and control between week 0 and week 12, where the change or values of HOMA-IR index were calculated according to the formula: [Fasting blood glucose (mmol/l)×Fasting blood insulin (mIU/l)]/22.5, for the HFD-fed rAAV-GPR110 or rAAV-GFP mice at the end point, i.e., week 12, upon i.v. injection;

FIG. 2J shows results of glucose tolerance test (GTT) on rAAV-GPR110 mice and control in terms of serum glucose level over body weight (1 g/kg) (left panel) and area under curve (AUC) (right panel) measured at week 10 upon i.v. injection;

FIG. 2K shows results of pyruvate tolerance test (PTT) on rAAV-GPR110 mice and control in terms of serum glucose level over body weight (1 g/kg) (left panel) and area under curve (AUC) (right panel) measured at week 11 upon i.v. injection;

FIG. 2L shows results of insulin tolerance test (ITT) on rAAV-GPR110 mice and control in terms of serum glucose level over body weight (0.5 U/kg) (left panel) and area under curve (AUC) (right panel) measured at week 12 upon i.v. injection.

FIGS. 3A-3J show that a hepatic overexpression of GPR110 in a mouse model exhibits mild metabolic abnormalities, in which data represents as mean±SEM; repeated with three independent experiments; P value analyzed by two-tailed Student's t test. *P<0.05, **P<0.01, ***P<0.001:

FIG. 3A schematically depicts a scheme of treatments to the mouse model according to certain embodiments, where 8-week-old male C57BL/6J mice were infected with 3×10¹¹ copies of AAV encoding GPR110 (rAAV-GPR110, i.v.) or control (rAAV-GFP, i.v.) after feeding STC for 8 weeks;

FIG. 3B shows hepatic mRNA expression levels of GPR110 from STC-fed mice with liver-specific GPR110 overexpression as determined by RT-qPCR analysis;

FIG. 3C shows an immunoblotting analysis of hepatic protein expression level of GPR110 from STC-fed mice liver with GPR110 overexpression (left panel) and quantification of hepatic protein expression levels of GPR110 (right panel), where each lane is a sample from a different individual; n=3 per group;

FIG. 3D shows the change of body weight in rAAV-GPR110 mice and control biweekly upon i.v. injection;

FIG. 3E shows the change of fasting blood glucose in rAAV-GPR110 mice and control biweekly upon i.v. injection;

FIG. 3F shows fasting blood insulin level in rAAV-GPR110 mice and control between week 0 and week 13;

FIG. 3G shows HOMA-IR values of rAAV-GPR110 mice and control measured and calculated at the end point, i.e., week 13;

FIG. 3H shows results of GTT on rAAV-GPR110 mice and control in terms of serum glucose over body weight (1 g/kg) (left panel) and area under curve (AUC) (right panel) measured at week 10 upon i.v. injection;

FIG. 3I shows results of PTT on rAAV-GPR110 mice and control in terms of serum glucose over body weight (1 g/kg) (left panel) and AUC (right panel) measured at week 11 upon i.v. injection;

FIG. 3J shows results of ITT on rAAV-GPR110 mice and control in terms of serum glucose over body weight (0.5 U/kg) (left panel) and AUC (right panel) measured at week 12 upon i.v. injection.

FIGS. 4A-4G show that overexpression of GPR110 did not interfere adipose tissues and other metabolic phenotypes, in which data represents as mean±SEM; repeated with three independent experiments; P value analyzed by two-tailed Student's t test. *P<0.05, **P<0.01, ***P<0.001:

FIG. 4A shows mRNA expression levels of GPR110 in subcutaneous adipose tissue (SWAT), epididymal white adipose tissue (EWAT), and brown adipose tissue (BAT) of rAAV-GPR110 mice and control;

FIG. 4B shows an average daily pedestrian locomotion of rAAV-GPR110 mice and control at daytime (Light), nighttime (Dark), and overall daily (Full day);

FIG. 4C shows an average distance in cage locomotion of rAAV-GPR110 mice and control at daytime (Light), nighttime (Dark), and overall daily (Full day);

FIG. 4D shows an average energy expenditure of rAAV-GPR110 mice and control at daytime (Light), nighttime (Dark), and overall daily (Full day);

FIG. 4E shows an average daily food intake by rAAV-GPR110 mice and control at daytime (Light), nighttime (Dark), and overall daily (Full day);

FIG. 4F shows an average daily water intake by rAAV-GPR110 mice and control at daytime (Light), nighttime (Dark), and overall daily (Full day);

FIG. 4G shows respiratory exchange ratio of rAAV-GPR110 mice and control at daytime (Light), nighttime (Dark), and overall daily (Full day).

FIGS. 5A-5L show that deletion of hepatic GPR110 protects against diet-induced glucose intolerance in GPR110-overexpressed mice, in which data represents as mean±SEM; repeated with three independent experiments; P value analyzed by two-tailed Student's t test. *P<0.05, **P<0.01, ***P<0.001:

FIG. 5A schematically depicts a scheme of treatments to different groups of mice according to certain embodiments, where 8-week-old male C57BL/6J mice were initially received HFD for 8 weeks before infected with either 3×10¹¹ copies of rAAV encoding GPR110 (rAAV-GPR110, i.v.) or control (rAAV-GFP, i.v.), and after 2 weeks further infected with two different sequences of GPR110 antisense oligonucleotides (ASO1-GPR110, ASO2-GPR110) each at 5 mg/kg, one dose per week, administered via subcutaneous injection (s.c.), or scrambled control via s.c. (ASO-NC);

FIG. 5B shows hepatic mRNA expression levels of GPR110 in different treatment groups of mice (GFP-NC, GPR110-NC, GPR110-ASO1 or GPR110-ASO2) fed with HFD, respectively, as determined by RT-qPCR analysis, where mRNA expression levels of the target genes were normalized to the expression of mouse GAPDH. rAAV-NC group was set as 1 for fold-change calculation; n=8 per group;

FIG. 5C shows immunoblotting analyses of GPR110 and 13-tubulin from livers of HFD-fed, rAAV-GFP or rAAV-GPR110 mice treated with either ASO-NC or ASO-GPR110 (left panel), where each lane is a sample from a different individual; and quantification of protein expression levels of GPR110 and 13-tubulin (right panel), where protein expression levels were normalized to the expression of 13-tubulin;

FIG. 5D shows the change of body weight in different treatment groups of mice biweekly over time upon i.v. injection of rAAV-GPR110 or rAAV-GFP;

FIG. 5E shows the percentage change of fat mass in different treatment groups of mice between week 0 and week 12 upon i.v. injection of rAAV-GPR110 or rAAV-GFP;

FIG. 5F shows the percentage change of lean mass in different treatment groups of mice between week 0 and week 12 upon i.v. injection of rAAV-GPR110 or rAAV-GFP;

FIG. 5G shows the fasting blood glucose levels in different treatment groups of mice biweekly upon i.v. injection of rAAV-GPR110 or rAAV-GFP;

FIG. 5H shows the fasting serum insulin levels in different treatment groups of mice at the end point upon i.v. injection of rAAV-GPR110 or rAAV-GFP;

FIG. 5I shows HOMA-IR values in different treatment groups of mice at the end point upon i.v. injection of rAAV-GPR110 or rAAV-GFP according to the formula: [Fasting blood glucose (mmol/l)×Fasting blood insulin (mIU/l)]/22.5, for the HFD-fed rAAV-GPR110 or rAAV-GFP mice;

FIG. 5J shows results of GTT on different treatment groups in terms of serum glucose over body weight (1 g/kg) (left panel) and AUC (right) measured at week 10 upon i.v. injection of rAAV-GPR110 or rAAV-GFP;

FIG. 5K shows results of PTT on different treatment groups in terms of serum glucose over body weight (1 g/kg) (left panel) and AUC (right) measured at week 11 upon i.v. injection of rAAV-GPR110 or rAAV-GFP;

FIG. 5L shows results of ITT on different treatment groups in terms of serum glucose over body weight (0.5 U/kg) (left panel) and AUC (right) measured at week 12 upon i.v. injection of rAAV-GPR110 or rAAV-GFP.

FIGS. 6A-6J show that a hepatic knockdown of GPR110 in STC-fed mice does not exhibit metabolic abnormalities, in which data represents as mean±SEM; n=8 mice per group; repeated with three independent experiments; P value analyzed by two-tailed Student's t test. *P<0.05, **P<0.01, ***P<0.001:

FIG. 6A schematically depicts a scheme of treatments to a mouse model according to certain embodiments, where 8-week-old male C57BL/6J mice were infected with two different sequences of GPR110 ASO (ASO1-GPR110 and ASO2-GPR110) each at 5 mg/kg, one dose per week via s.c., or scrambled control (ASO-NC) via s.c.;

FIG. 6B shows mRNA expression levels of GPR110 in liver and kidney as determined by RT-qPCR analysis, where mRNA expression levels of GPR110 in different tissues were normalized to the expression of mouse GAPDH;

FIG. 6C shows immunoblotting analysis of hepatic protein expression level of GPR110 from STC-fed mice liver with GPR110 knockdown (left panel) and quantification of hepatic protein expression levels of GPR110 (right panel), where each lane is a sample from a different individual;

FIG. 6D shows the change of body weight in different treatment groups biweekly upon s.c. injection of ASO or ASO-NC;

FIG. 6E shows the fasting blood glucose level in different treatment groups biweekly upon s.c. injection of ASO or ASO-NC;

FIG. 6F shows the fasting blood insulin level in different treatment groups biweekly upon s.c. injection of ASO or ASO-NC;

FIG. 6G shows HOMA-IR values in different treatment groups measured and calculated at the end point, i.e., week 13;

FIG. 6H shows results of GTT on different treatment groups in terms of serum glucose over body weight (1 g/kg) (left panel) and AUC (right) measured at week 10 upon s.c. injection of ASO or ASO-NC;

FIG. 6I shows results of PTT on different treatment groups in terms of serum glucose over body weight (1 g/kg) (left panel) and AUC (right) measured at week 11 upon s.c. injection of ASO or ASO-NC;

FIG. 6J shows results of ITT on different treatment groups in terms of serum glucose over body weight (0.5 U/kg) (left panel) and AUC (right) measured at week 12 upon s.c. injection of ASO or ASO-NC.

FIGS. 7A-7J show that Up-regulation of hepatic GPR110 exaggerates liver steatosis in HFD mice fed with HFD while down-regulation of hepatic GPR110 protects mice from diet-induced liver lipid accumulation, where 8-week-old male C57BL/6J mice were infected with either 3×10¹¹ copies of rAAV encoding GPR110 (rAAV-GPR110) via i.v. or control (rAAV-NC) via i.v. and then two different sequences of GPR110 antisense oligonucleotides (ASO1-GPR110, ASO2-GPR110) each at 5 mg/kg, one dose per week, via s.c. or scrambled control (ASO-NC) via s.c., after 8-week HFD feeding, in which the data shown represents as mean±SEM; n=8 per group; repeated with three independent experiments; P value analyzed by two-tailed Student's t test. *P<0.05, **P<0.01, ***P<0.001:

FIG. 7A shows serum cholesterol (CHO) in different treatment groups at week 13 upon i.v. injection of rAAV-GPR110 or rAAV-NC;

FIG. 7B shows serum triglyceride (TG) in different treatment groups at week 13 upon i.v. injection of rAAV-GPR110 or rAAV-NC;

FIG. 7C shows serum free fatty acid (FFA) levels in different treatment groups measured at week 13 upon i.v. injection of rAAV-GPR110 or rAAV-NC;

FIG. 7D shows serum high-density lipoprotein (HDL) and low-density lipoprotein (LDL) in different treatment groups measured at week 13 upon i.v. injection of rAAV-GPR110 or rAAV-NC;

FIG. 7E shows levels of serum aspartate transaminase (AST) and alanine transaminase (ALT) in different treatment groups measured at week 13 upon i.v. injection of rAAV-GPR110 or rAAV-NC;

FIG. 7F shows the ratio of the liver weight to the body weight calculated after sacrificing the mice from different treatment groups;

FIG. 7G shows representative gross pictures of liver tissues (first row of left panel) and images of H&E stained sections (second row of left panel), Oil Red O stained sections (third row of left panel), and Masson's trichrome stained sections (fourth row of left panel), where scale bar is 200 μm; and the percentage of lipid area according to the H&E stained sections (right panel);

FIG. 7H shows hepatic cholesterol (CHO) in different treatment groups at week 13 upon i.v. injection of rAAV-GPR110 or rAAV-NC;

FIG. 7I shows hepatic triglyceride (TG) in different treatment groups at week 13 upon i.v. injection of rAAV-GPR110 or rAAV-NC;

FIG. 7J shows hepatic free fatty acid (FFA) in different treatment groups, which were normalized by the weight of liver samples used for lipid extraction.

FIGS. 8A-8D shows that GPR110 is a major regulator of hepatic lipid metabolism, where 8-week-old male C57BL/6J mice were infected with either 3×10¹¹ copies of rAAV encoding GPR110 (rAAV-GPR110) via i.v. or control (rAAV-NC) via i.v. and then two different sequences of GPR110 antisense oligonucleotides (ASO1-GPR110, ASO2-GPR110) each at 5 mg/kg, one dose per week, via s.c. or scrambled control (ASO-NC) via s.c., after HFD feeding, in which the data shown represents as mean±SEM; n=8 per group; repeated with three independent experiments; P value analyzed by two-tailed Student's t test. *P<0.05, **P<0.01, ***P<0.001:

FIG. 8A shows results of Kyoto Encyclopedia of Genes and Genomes (KEGG) pathway assay of differential mRNA transcripts in rAAV-GPR110 and ASO-treated groups identified by RNA sequencing (RNA- seq);

FIG. 8B shows a heat map in log₂ scale of fold change in the expression levels of a set of genes (left side of the heat map) involved in lipid metabolism from the RNA-seq data of livers in the HFD-fed mice infected by rAAV-GPR110 or rAAV-GPR110 followed by ASO1-treatment, where n=3 per group;

FIG. 8C shows mRNA expression levels of genes according to the heatmap in FIG. 8B from different groups of mice received either GFP-NC, GPR110-NC, GPR110-ASO1 or GPR110-ASO2 fed with HFD, respectively, as determined by RT-qPCR analysis, where n=6 per group;

FIG. 8D shows de novo lipogenic activity in different treatment groups in terms of the measured ³H labeling of lipogenic Acetyl-CoA from 0.5 μCi ³H-acetate.

FIGS. 9A-9G show that SCD1 expression is regulated by GPR110 in primary hepatocytes, in which data represents as mean±SEM; repeated with three independent experiments; P value analysed by two-tailed Student's t test. *P<0.05, **P<0.01, ***P<0.001:

FIG. 9A shows mRNA expression levels of GPR110 and SCD1 from different groups were assessed, as determined by RT-qPCR analysis, where the primary hepatocytes isolated from 8-week-old male C57BL/6J mice fed with STC were infected with either adenoviral vector expressing GPR110 (ADV-GPR110) or control adenovirus expressing GFP (ADV-GFP) 24 h after plating, followed by transfection with ASO1-GPR110, ASO2-GPR110 or ASO-NC for another 6 hours;

FIG. 9B shows an immunoblotting analysis for the expression level of GPR110 and SCD1 from different groups of primary hepatocytes from STC-fed mice depicted in FIG. 9A (upper panel) and quantification of protein expression levels of GPR110 and SCD1 (lower panel), where protein expression levels were normalized to the expression of 13-tubulin; each lane is a sample from a different plate; n=3 per group; the samples for GFP were set as 1 for fold-change calculation;

FIG. 9C shows results of luciferase assay on cell lysates from HEK293 cells which were infected with pGL3-SCD1 promoter-luciferase plasmid and adenoviral vector expressing GPR110 (ADV-GPR110) or GFP (ADV-GFP) for 48 h and DHEA was added to the transfected cells at the concentration of 100 μM for 24 h;

FIG. 9D shows results of RT-qPCR analysis on the cell lysates from HEK293 cells infected with different plasmid and viral vector depicted in FIG. 9C;

FIG. 9E shows intracellular cholesterol (CHO) extracted from primary mouse hepatocytes infected with either adenoviral vector expressing GPR110 (ADV-GPR110) or control ADV-GFP, followed by transfecting with scramble or shSCD1-1 or shSCD1-2 plasmids for another 72 hours;

FIG. 9F shows intracellular triglyceride (TG) extracted from different treatment groups of primary mouse hepatocytes depicted in FIG. 9E;

FIG. 9G shows intracellular free fatty acid (FFA) extracted from different treatment groups of primary mouse hepatocytes depicted in FIG. 9E.

FIGS. 10A-10J show that an inhibition of SCD1 alleviates the glucose impairment in mice with hepatic GPR110 overexpression, in which the data represents as mean±SEM; n=8 per group; repeated with three independent experiments; P value analysed by two-tailed Student's t test. *P<0.05, **P<0.01, ***P<0.001:

FIG. 10A schematically depicts a scheme of different treatments to a mouse model according to certain embodiments, where eight-week-old male C57BL/6J mice were infected with either 3×10¹¹ copies of rAAV encoding GPR110 (rAAV-GPR110) via i.v. or control (rAAV-GFP) via i.v. after 8-week HFD feeding, and then 2 weeks after infection treated with SCD1 inhibitor, MK8245, at 10 mg/kg/week via oral administration (p.o.) or inhibitor vehicle (inhibitor-Veh.) via p.o.;

FIG. 10B shows hepatic mRNA expression levels of GPR110 from different groups of HFD-fed mice treated with rAAV and inhibitor, respectively, as determined by RT-qPCR analysis, where mRNA expression levels of the target genes were normalized to the expression of mouse GAPDH;

FIG. 10C shows an immunoblotting analysis for the hepatic protein expression level of GPR110 and SCD1 from different groups of HFD-fed mice;

FIG. 10D shows the change of body weight in different treatment groups of mice measured biweekly upon i.v. injection of rAAV-GPR110 or rAAV-GFP and then SCD1 inhibitor via p.o.;

FIG. 10E shows the fasting blood glucose level in different treatment groups of mice measured biweekly upon i.v. injection of rAAV-GPR110 or rAAV-GFP and then SCD1 inhibitor via p.o.;

FIG. 10F shows the fasting serum insulin level in different treatment groups of mice measured biweekly upon i.v. injection of rAAV-GPR110 or rAAV-GFP and then SCD1 inhibitor via p.o.;

FIG. 10G shows HOMA-IR values according to the formula: [Fasting blood glucose (mmol/l)×Fasting blood insulin (mIU/l)]/22.5 for different treatment groups of mice measured and calculated at the end point, i.e., week 13, upon i.v. injection of rAAV GPR110 or rAAV-GFP and then SCD1 inhibitor via p.o.;

FIG. 10H shows results of GTT on different treatment groups in terms of serum glucose over body weight (1 g/kg) (left panel) and AUC (right) measured at week 10 upon i.v. injection of rAAV GPR110 or rAAV-GFP and then SCD1 inhibitor via p.o.;

FIG. 10I shows results of PTT on different treatment groups in terms of serum glucose over body weight (1 g/kg) (left panel) and AUC (right) measured at week 11 upon i.v. injection of rAAV GPR110 or rAAV-GFP and then SCD1 inhibitor via p.o.;

FIG. 10J shows results of ITT on different treatment groups in terms of serum glucose over body weight (0.5 U/kg) (left panel) and AUC (right) measured at week 12 upon i.v. injection of rAAV GPR110 or rAAV-GFP and then SCD1 inhibitor via p.o.;

FIGS. 11A-11J show that an inhibition of hepatic SCD1 partially alleviates the severity of hepatic steatosis in GPR110 overexpression mice, where eight-week-old male C57BL/6J mice were infected with either 3×10¹¹ copies of rAAV encoding GPR110 (rAAV-GPR110) via i.v. or control (rAAV-GFP) via i.v. after 8-week HFD feeding, and then 2 weeks after infection treated with SCD1 inhibitor, MK8245, at 10 mg/kg/week via oral administration (p.o.) or inhibitor vehicle (inhibitor-Veh.) via p.o., in which Data represents as mean±SEM; n=8 per group; repeated with three independent experiments; P value analyzed by two-tailed Student's t test. *P<0.05, **P<0.01, ***P<0.001:

FIG. 11A shows serum cholesterol (CHO) in different treatment groups at week 13 upon i.v. injection of rAAV-GPR110 or rAAV-GFP and then SCD1 inhibitor via p.o.;

FIG. 11B shows serum triglyceride (TG) in different treatment groups at week 13 upon i.v. injection of rAAV-GPR110 or rAAV-NC and then SCD1 inhibitor via p.o.;

FIG. 11C shows serum free fatty acid (FFA) levels in different treatment groups measured at week 13 upon i.v. injection of rAAV-GPR110 or rAAV-NC and then SCD1 inhibitor via p.o.;

FIG. 11D shows serum high-density lipoprotein (HDL) and low-density lipoprotein (LDL) in different treatment groups measured at week 13 upon i.v. injection of rAAV-GPR110 or rAAV-NC and then SCD1 inhibitor via p.o.;

FIG. 11E shows levels of serum aspartate transaminase (AST) and alanine transaminase (ALT) in different treatment groups measured at week 13 upon i.v. injection of rAAV-GPR110 or rAAV-NC and then SCD1 inhibitor via p.o.;

FIG. 11F shows the ratio of the liver weight to the body weight calculated after sacrificing the mice from different treatment groups;

FIG. 11G shows representative gross pictures of liver tissues (first row of left panel) and images of H&E stained sections (second row of left panel), Oil Red Ostained sections (third row of left panel), and Masson's trichrome stained sections (fourth row of left panel), where scale bar is 200 μm; and the percentage of lipid area according to the H&E stained sections (right panel);

FIG. 11H shows hepatic cholesterol (CHO) in different treatment groups at week 13 upon i.v. injection of rAAV-GPR110 or rAAV-NC and then SCD1 inhibitor via p.o.;

FIG. 11I shows hepatic triglyceride (TG) in different treatment groups at week 13 upon i.v. injection of rAAV-GPR110 or rAAV-NC and then SCD1 inhibitor via p.o.;

FIG. 11J shows hepatic free fatty acid (FFA) in different treatment groups, which were normalized by the weight of liver samples used for lipid extraction.

FIGS. 12A-12G show that hepatic expression of GPR110 is upregulated in obese patients with hepatic steatosis when compared to those with normal liver morphology, which is positively associated with hepatic SCD1 expression level, where data represents as mean±SEM P value analyzed by two-tailed Student's t test. *P<0.05, **P<0.01, ***P<0.001:

FIG. 12A shows normalized log₂ mRNA expression of GPR110 in lean people without NAFLD (n=12), obese people without NAFLD (n=17) or obese patients with NAFLD (n=8) according to the Gene Expression Omnibus (GEO) database (GEO Profile # GDS4881/8126820);

FIG. 12B shows a correlation between GPR110 and SCD1 in liver of human subjects based on the GEO database;

FIG. 12C shows images of liver tissues with H&E staining (the top row in upper panel) and immunohistochemical staining (IHC) (the bottom row in upper panel) of GPR110 from patients with different degree of NAFLD (scale bar: 200 μm); and the percentage of GPR110 positive area according to H&E stained and IHC stained images (lower panel) (n=3 per group);

FIG. 12D shows normalized Log₂ mRNA expression of IL-1β in lean people without NAFLD (n=12), obese subjects without NAFLD (n=17) or obese patients with NAFLD (n=8) according to the GEO database (GEO Profile # GDS4881/8126820);

FIG. 12E shows hepatic mRNA expression levels of IL-1β in in STC-fed mice (lean) or HFD-fed mice treated with CC14 or STZ as determined by RT-qPCR;

FIG. 12F shows hepatic mRNA expression levels of GPR110 in in STC-fed mice (lean) or HFD-fed mice treated with CC14 or STZ as determined by RT-qPCR;

FIG. 12G shows mRNA expression levels of SCD1 in STC-fed mice (lean) or HFD-fed mice treated with CC14 or STZ as determined by RT-qPCR.

FIG. 13 shows the difference in GPR110 mRNA levels in four different human liver cell lines transfected by four different small interfering RNA (siRNA) according to certain embodiments.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure has uncovered a previously unrecognized role of GPR110 in regulating hepatic lipid metabolism. Firstly, GPR110 is highly expressed in liver of healthy subjects, and hepatic GPR110 (an amino acid sequence of human hepatic GPR110 and mouse hepatic GPR110 are represented by SEQ ID NOs: 1 and 2, respectively) is required for regulating lipid content in liver of diet-induced obese mice by both gain-of-function and loss-of-function approaches. Secondly, the hepatic GPR110 expression level of healthy obese human and mouse are downregulated. The downregulation of hepatic GPR110 expression level in obese subjects is believed to be a protective mechanism to prevent over-accumulation of lipid in liver. In some examples described herein, HFD-induced steatosis and liver injury are shown to be exacerbated in obese mice with high GPR110 overexpression level, and knockdown hepatic GPR110 alleviated the severity of obesity-induced NAFLD.

Subsequently, RNA-sequencing analysis is performed to verify the GPR110's role in regulating lipogenesis. The expression levels of SCD1 mRNAs and protein are shown to be dramatically upregulated in the livers of rAAV-GPR110 mice and repressed in GPR110-ASOs treated rAAV-GPR110 mice, where SCD1 is the rate-limiting enzyme that catalyzes the conversion of saturated long-chain fatty acids into monounsaturated fatty acids, and its expression is tightly regulated by various parameters, including hormonal and nutrient factors.

GPR110 is known to mediate palmitic acids to activate the mTOR and SREBP1 pathways to promote fat synthesis in mammary gland tissues, where SREBP1 is the key transcription factor for regulating SCD1 gene expression. RNA sequencing analysis in the present disclosure also reveals that the expression of SREBP1 mRNA is dramatically upregulated in the livers of rAAV-GPR110 mice and repressed in GPR110-ASOs treated rAAV-GPR110 mice, indicating that GPR110 may regulate SCD1 expression via SREBP1 in the liver. Importantly, the expression of hepatic GPR110 and SCD1 mRNAs can be further increased in the livers of subjects with more severe NAFLD, contributing to its acceleration and aggravation.

To verify that the up-regulation of hepatic SCD1 expression levels is responsible for the changes in metabolic phenotype in the rAAV-GPR110 mice, the present disclosure also provides both in vivo and in vitro experimental data obtained from using SCD1 shRNAs and inhibitors. It is found that pharmacologically inhibiting SCD1 by the SCD1 inhibitor, 5-[3-[4-(2-bromo-5-fluorophenoxy)- 1-piperidinyl]-5-isoxazolyl]-2H-tetrazole-2-acetic acid (C₁₇H₁₆BrFN₆O₄) or called MK8245, is sufficient to rescue the key metabolic dysregulations caused by GPR110 overexpression in the liver. These results strongly support the conclusion that GPR110 induces SCD1 expression, leading to an increase in de novo lipogenesis in the liver and exacerbating obese-induced NAFLD.

The present disclosure also provides that global knockout of SCD1 improves insulin sensitivity, higher-energy metabolism, and more resistant to diet-induced obesity in a mouse model by the activation of lipid oxidation, in addition to the reduction of triglyceride synthesis and storage. In addition, storage of triglyceride is significantly reduced and a lower level of very low-density lipoprotein (VLDL) is produced in the ob/ob mice with SCD1 mutations. Remarkably, liver-specific KO of SCD1 is sufficient to reduce high-carbohydrate diet-induced adiposity with a significant reduction of hepatic lipogenesis and improved glucose tolerance. SCD1 inhibition is known to be a therapeutic strategy for the treatment of metabolic syndrome.

However, SCD1 is highly expressed in various tissues, especially adipose tissues. Also, expression level and activity of SCD1 is very tightly regulated. Harmful consequences from inhibiting SCD1 have been reported, such as the inhibition of fat mobilization in adipose tissues and the promotion of proinflammatory and endoplasmic reticulum stress by accumulation of SCD1 substrates, suggesting that optimal level of SCD1 is required to maintain health.

In contrast to SCD1, the hepatic GPR110 may be dispensable in adults based on a dramatical reduction of GPR110 in the livers of HFD-fed mice and health obese subjects described in the present disclosure. Therefore, targeting hepatic GPR110 is a potentially safe or viable treatment of NAFLD.

In accordance with the RNA-sequencing analysis results described herein, repressing GPR110 can also regulate the expression of many other lipid metabolism genes. The ASO-based strategy used in various embodiments is an approach to knockdown the expression of GPR110 in liver, alternative to using GPR110 antagonist which has not been proven.

Since the amino acid sequence of GPR110 is highly conserved in humans and mice, the in vivo data obtained from the mouse model in the present disclosure should be of high relevance to future drug development for treating NAFLD or associated conditions/symptoms in human beings.

The following examples are intended to assist the understanding of the present invention without limiting effect. The scope of the invention should be defined in the appended claims.

### EXAMPLES

### Example 1—GPR110 Mainly Expresses in Liver of Mice and its Expression is Downregulated after HFD Treatment

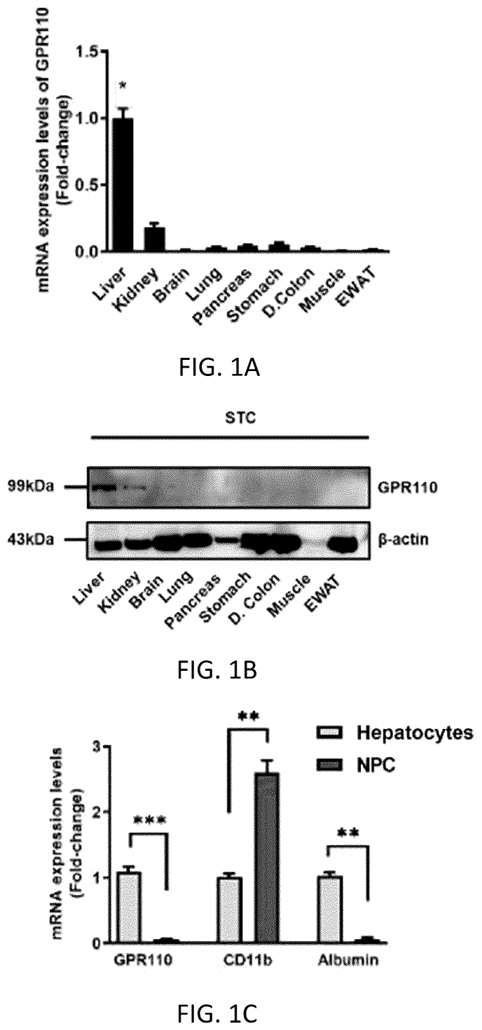

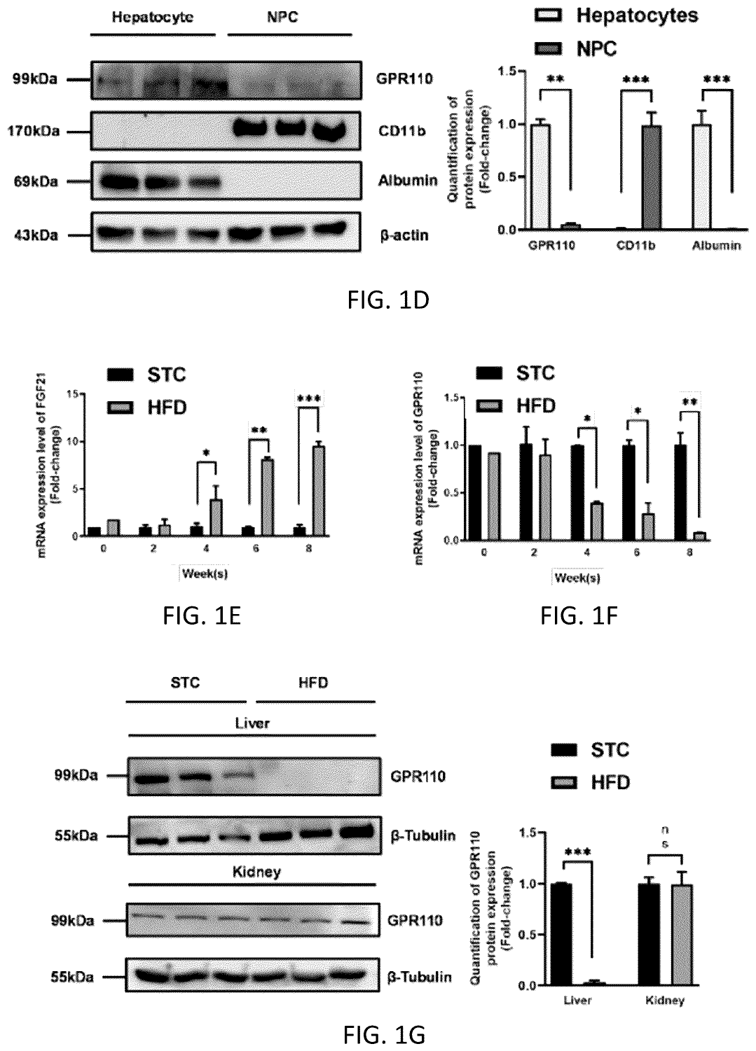

Microarray analysis was used to examine the change in expression levels of hepatic GPCRs in mice after HFD treatment (Table 1). In this example, it is found that GPR110 was mainly expressed in liver and its expression was dramatically decreased in the HFD-fed mice as compared to their STC-fed littermates. Remarkably, mRNA of GPR110 was mainly expressed in the liver of adult mice (FIG. 1A). GPR110 protein expression was also shown in those tissues by Western blot analysis. GRP110 proteins were mainly detected in liver and kidney samples (FIG. 1B).

TABLE 1

| Gene | | STC | | HFD | | |
|---|---|---|---|---|---|---|
| Symbol | Gene Description | average | SD | average | SD | p value |
| Htr1a | 5-hydroxytryptamine | 5.52 | 0.46 | 5.67 | 0.45 | 0.64018 |
| | (serotonin) receptor 1A | | | | | |
| Htr1b | 5-hydroxytryptamine | 6.27 | 0.34 | 5.97 | 0.17 | 0.13440 |
| | (serotonin) receptor 1B | | | | | |
| Htr1d | 5-hydroxytryptamine | 4.44 | 0.51 | 4.14 | 0.52 | 0.50166 |
| | (serotonin) receptor 1D | | | | | |
| Htr1f | 5-hydroxytryptamine | 5.94 | 0.29 | 6.03 | 0.29 | 0.60580 |
| | (serotonin) receptor 1F | | | | | |
| Htr2a | 5-hydroxytryptamine | 6.47 | 0.34 | 6.74 | 0.37 | 0.36038 |
| | (serotonin) receptor 2A | | | | | |
| Htr2b | 5-hydroxytryptamine | 4.9 | 0.75 | 4.86 | 0.76 | 0.91875 |
| | (serotonin) receptor 2B | | | | | |
| Htr2c | 5-hydroxytryptamine | 8.03 | 0.31 | 8.22 | 0.38 | 0.38376 |
| | (serotonin) receptor 2C | | | | | |
| Htr3a | 5-hydroxytryptamine | 4.97 | 0.52 | 4.86 | 0.51 | 0.75293 |
| | (serotonin) receptor 3A | | | | | |
| Htr3b | 5-hydroxytryptamine | 5.81 | 0.83 | 5.75 | 0.89 | 0.90944 |
| | (serotonin) receptor 3B | | | | | |
| Htr5a | 5-hydroxytryptamine | 5.93 | 0.43 | 6.05 | 0.44 | 0.72994 |
| | (serotonin) receptor 5A | | | | | |
| Htr5b | 5-hydroxytryptamine | 4.49 | 0.15 | 5.28 | 0.15 | 0.41513 |
| | (serotonin) receptor 5B | | | | | |
| Htr6 | 5-hydroxytryptamine | 5.9 | 0.39 | 6.37 | 0.30 | 0.24233 |
| | (serotonin) receptor 6 | | | | | |
| Htr7 | 5-hydroxytryptamine | 4.63 | 0.50 | 4.95 | 0.46 | 0.42411 |
| | (serotonin) receptor 7 | | | | | |
| Adora1 | adenosine Al receptor | 7.07 | 1.04 | 8.51 | 0.64 | 0.00637 |
| Adora2a | adenosine A2a receptor | 5.15 | 0.18 | 5.44 | 0.16 | 0.32188 |
| Adora2b | adenosine A2b receptor | 4.74 | 0.85 | 5.26 | 0.61 | 0.32740 |
| Adora3 | adenosine A3 receptor | 4.96 | 0.47 | 5 | 0.48 | 0.90179 |
| Adora3 | adenosine A3 receptor | 3.94 | 0.96 | 4.36 | 1.07 | 0.49665 |
| Adra1a | adrenergic receptor, alpha 1a | 6.36 | 0.62 | 6.99 | 0.47 | 0.04982 |
| Adra1b | adrenergic receptor, alpha 1b | 8.33 | 0.62 | 8.5 | 0.65 | 0.66073 |
| Adra1d | adrenergic receptor, alpha 1d | 5.94 | 0.39 | 6.45 | 0.31 | 0.18180 |
| Adra2a | adrenergic receptor, alpha 2a | 5.95 | 0.38 | 6.12 | 0.40 | 0.54876 |
| Adra2b | adrenergic receptor, alpha 2b | 8.09 | 0.63 | 8.81 | 0.58 | 0.03652 |
| Adra2c | adrenergic receptor, alpha 2c | 8.02 | 0.53 | 8.26 | 0.43 | 0.53429 |
| Adrb1 | adrenergic receptor, beta 1 | 6.48 | 0.45 | 7.02 | 0.43 | 0.34887 |
| Adrb2 | adrenergic receptor, beta 2 | 5.54 | 0.80 | 5.84 | 0.72 | 0.44498 |
| Adrb3 | adrenergic receptor, beta 3 | 4.02 | 0.81 | 4.07 | 0.88 | 0.91950 |
| Agtr1a | angiotensin II receptor, type | 9.65 | 0.26 | 9.74 | 0.24 | 0.51518 |
| | 1a | | | | | |
| Agtr1b | angiotensin II receptor, type | 5.32 | 0.32 | 5.74 | 0.30 | 0.05626 |
| | 1b | | | | | |
| Aplnr | apelin receptor | 4.98 | 1.00 | 5.61 | 0.31 | 0.19545 |
| Avpr1a | arginine vasopressin receptor | 7.3 | 0.61 | 6.76 | 0.42 | 0.05483 |
| | 1A | | | | | |
| Avpr2 | arginine vasopressin receptor | 3.37 | 0.49 | 4.03 | 0.64 | 0.40725 |
| | 2 | | | | | |
| Brs3 | bombesin-like receptor 3 | 6.09 | 0.47 | 6.02 | 0.46 | 0.83354 |
| Bdkrb1 | bradykinin receptor, beta 1 | 4.99 | 0.50 | 5.1 | 0.58 | 0.82810 |
| Bdkrb2 | bradykinin receptor, beta 2 | 5.59 | 0.59 | 5.83 | 0.48 | 0.41334 |
| Cnr1 | cannabinoid receptor 1 | 7.86 | 0.47 | 7.59 | 0.44 | 0.30064 |
| | (brain) | | | | | |
| Cnr2 | cannabinoid receptor 2 | 4.53 | 0.39 | 4.46 | 0.33 | 0.80140 |
| | (macrophage) | | | | | |
| Xcr1 | chemokine (C motif) receptor | 5.38 | 0.20 | 5.46 | 0.58 | 0.81747 |
| | 1 | | | | | |
| Ccr1 | chemokine (C-C motif) | 3.69 | 0.86 | 2.87 | 0.84 | 0.12177 |
| | receptor 1 | | | | | |
| Ccr10 | chemokine (C-C motif) | 4.16 | 1.08 | 4.48 | 1.10 | 0.65062 |
| | receptor 10 | | | | | |
| Ccr2 | chemokine (C-C motif) | 4.86 | 0.42 | 5.48 | 0.38 | 0.01481 |
| | receptor 2 | | | | | |
| Ccr3 | chemokine (C-C motif) | 2.85 | 0.80 | 3.43 | 0.83 | 0.47266 |
| | receptor 3 | | | | | |
| Ccr4 | chemokine (C-C motif) | 0.42 | 1.66 | 1.81 | 1.51 | 0.11360 |
| | receptor 4 | | | | | |
| Ccr5 | chemokine (C-C motif) | 6.13 | 0.29 | 6.38 | 0.35 | 0.49865 |
| | receptor 5 | | | | | |
| Ccr6 | chemokine (C-C motif) | 4.71 | 0.44 | 5.02 | 0.46 | 0.25030 |
| | receptor 6 | | | | | |
| Ccr7 | chemokine (C-C motif) | 4.75 | 0.41 | 5.2 | 0.42 | 0.39125 |
| | receptor 7 | | | | | |
| Ccr9 | chemokine (C-C motif) | 5.61 | 0.11 | 5.44 | 0.14 | 0.14825 |
| | receptor 9 | | | | | |
| Ccrl1 | chemokine (C-C motif) | 5.49 | 0.36 | 5.6 | 0.37 | 0.67156 |
| | receptor-like 1 | | | | | |
| Ccrl2 | chemokine (C-C motif) | 5.43 | 0.52 | 5.19 | 0.68 | 0.51424 |
| | receptor-like 2 | | | | | |
| Cx3cr1 | chemokine (C-X3-C) receptor | 5.26 | 0.74 | 5.27 | 0.76 | 0.98819 |
| | 1 | | | | | |
| Cxcr1 | chemokine (C-X-C motif) | 5.9 | 0.88 | 5.4 | 0.71 | 0.23663 |
| | receptor 1 | | | | | |
| Cxcr2 | chemokine (C-X-C motif) | 6.17 | 0.51 | 5.91 | 0.38 | 0.28316 |
| | receptor 2 | | | | | |
| Cxcr3 | chemokine (C-X-C motif) | 4.19 | 0.86 | 3.81 | 0.86 | 0.37835 |
| | receptor 3 | | | | | |
| Cxcr4 | chemokine (C-X-C motif) | 5.26 | 0.30 | 5.38 | 0.32 | 0.49019 |
| | receptor 4 | | | | | |
| Cxcr6 | chemokine (C-X-C motif) | 4.62 | 0.29 | 4.37 | 0.29 | 0.36634 |
| | receptor 6 | | | | | |
| Cxcr7 | chemokine (C-X-C motif) | 5.11 | 0.22 | 5.54 | 0.21 | 0.24448 |
| | receptor 7 | | | | | |
| Cmklr1 | chemokine-like receptor 1 | 6.52 | 0.26 | 6.59 | 0.27 | 0.65239 |
| Cckar | cholecystokinin A receptor | 5.15 | 0.53 | 5.99 | 0.25 | 0.02900 |
| Cckbr | cholecystokinin B receptor | 5.27 | 0.59 | 5.76 | 0.48 | 0.20122 |
| Chrm1 | cholinergic receptor, | 5.28 | 0.32 | 5.79 | 0.32 | 0.24786 |
| | muscarinic 1, CNS | | | | | |
| Chrm2 | cholinergic receptor, | 3 | 1.34 | 3.02 | 1.49 | 0.98491 |
| | muscarinic 2, cardiac | | | | | |
| Chrm3 | cholinergic receptor, | 5.55 | 0.62 | 6.44 | 0.54 | 0.01491 |
| | muscarinic 3, cardiac | | | | | |
| Chrm4 | cholinergic receptor, | 4.07 | 1.03 | 4.1 | 0.96 | 0.95456 |
| | muscarinic 4 | | | | | |
| Chrm5 | cholinergic receptor, | 4.6 | 0.43 | 4.65 | 0.42 | 0.92204 |
| | muscarinic 5 | | | | | |
| Chrna1 | cholinergic receptor, | 4.76 | 0.60 | 5.1 | 0.51 | 0.31847 |
| | nicotinic, alpha polypeptide 1 | | | | | |
| | (muscle) | | | | | |
| Chrna2 | cholinergic receptor, | 6.61 | 0.62 | 6.89 | 0.62 | 0.54576 |
| | nicotinic, alpha polypeptide 2 | | | | | |
| | (neuronal) | | | | | |
| Chrna3 | cholinergic receptor, | 6.22 | 0.42 | 6.35 | 0.39 | 0.54345 |
| | nicotinic, alpha polypeptide 3 | | | | | |
| Chrna4 | cholinergic receptor, | 5.45 | 0.46 | 5.47 | 0.41 | 0.95686 |
| | nicotinic, alpha polypeptide 4 | | | | | |
| Chrna5 | cholinergic receptor, | 4.74 | 0.61 | 5 | 0.49 | 0.45891 |
| | nicotinic, alpha polypeptide 5 | | | | | |
| Chrna6 | cholinergic receptor, | 3.66 | 1.48 | 2.93 | 1.41 | 0.33627 |
| | nicotinic, alpha polypeptide 6 | | | | | |
| Chrna7 | cholinergic receptor, | 8.97 | 0.24 | 9.15 | 0.32 | 0.32351 |
| | nicotinic, alpha polypeptide 7 | | | | | |
| Chrnb1 | cholinergic receptor, | 7.25 | 0.35 | 7.2 | 0.35 | 0.86077 |
| | nicotinic, beta polypeptide 1 | | | | | |
| | (muscle) | | | | | |
| Chrnb2 | cholinergic receptor, | 4.42 | 0.60 | 5.27 | 0.44 | 0.21835 |
| | nicotinic, beta polypeptide 2 | | | | | |
| | (neuronal) | | | | | |
| Chrnb3 | cholinergic receptor, | 4.23 | 0.50 | 4.4 | 0.50 | 0.53936 |
| | nicotinic, beta polypeptide 3 | | | | | |
| Chrnb4 | cholinergic receptor, | 6.09 | 0.22 | 6.4 | 0.21 | 0.15197 |
| | nicotinic, beta polypeptide 4 | | | | | |
| Chrnd | cholinergic receptor, | 5.86 | 0.52 | 6.3 | 0.62 | 0.30291 |
| | nicotinic, delta polypeptide | | | | | |
| Chrne | cholinergic receptor, | 5.18 | 0.26 | 5.22 | 0.39 | 0.90632 |
| | nicotinic, epsilon polypeptide | | | | | |
| Chrng | cholinergic receptor, | 5.79 | 0.55 | 6.04 | 0.54 | 0.45388 |
| | nicotinic, gamma polypeptide | | | | | |
| F2r | coagulation factor II | 9.13 | 0.78 | 9.12 | 0.79 | 0.97725 |
| | (thrombin) receptor | | | | | |
| F2rl1 | coagulation factor II | 3.73 | 0.55 | 4.71 | 0.38 | 0.05457 |
| | (thrombin) receptor-like 1 | | | | | |
| F2rl2 | coagulation factor II | 4.81 | 0.26 | 5.09 | 0.25 | 0.19600 |
| | (thrombin) receptor-like 2 | | | | | |
| F2rl3 | coagulation factor II | 5.77 | 0.46 | 5.81 | 0.46 | 0.90587 |
| | (thrombin) receptor-like 3 | | | | | |
| C3ar1 | complement component 3a | 6.59 | 0.49 | 7.06 | 0.52 | 0.10524 |
| | receptor 1 | | | | | |
| Cysltr1 | cysteinyl leukotriene receptor | 4.78 | 0.56 | 5.25 | 0.73 | 0.25262 |
| | 1 | | | | | |
| Cysltr2 | cysteinyl leukotriene receptor | 4.5 | 1.06 | 3.94 | 1.06 | 0.28245 |
| | 2 | | | | | |
| Drd1a | dopamine receptor D1A | 4.43 | 0.40 | 3.95 | 0.47 | 0.17215 |
| Drd2 | dopamine receptor D2 | 8.71 | 0.32 | 8.86 | 0.27 | 0.38864 |
| Drd3 | dopamine receptor D3 | 5.25 | 0.77 | 6.06 | 0.57 | 0.09265 |
| Drd4 | dopamine receptor D4 | 8.21 | 0.47 | 8.4 | 0.46 | 0.55598 |
| Ednra | endothelin receptor type A | 7.31 | 0.53 | 7.82 | 0.54 | 0.08885 |
| Ednrb | endothelin receptor type B | 7.72 | 0.38 | 8.29 | 0.43 | 0.04531 |
| Fshr | follicle stimulating hormone | 5.06 | 0.66 | 5.41 | 0.50 | 0.32926 |
| | receptor | | | | | |
| Fpr1 | formyl peptide receptor 1 | 6.7 | 0.23 | 6.88 | 0.32 | 0.51783 |
| Fpr2 | formyl peptide receptor 2 | 4.87 | 0.31 | 5.04 | 0.52 | 0.61891 |
| Ffar1 | free fatty acid receptor 1 | 3.57 | 0.89 | 3.78 | 0.94 | 0.66838 |
| Ffar2 | free fatty acid receptor 2 | 6.27 | 0.36 | 6.52 | 0.36 | 0.27399 |
| Gpbar1 | G protein-coupled bile acid | 5.49 | 0.63 | 5.11 | 0.55 | 0.40075 |
| | receptor 1 | | | | | |
| Gpr1 | G protein-coupled receptor 1 | 4.51 | 0.13 | 4.63 | 0.13 | 0.54999 |
| Gpr107 | G protein-coupled receptor | 7.34 | 0.52 | 7.54 | 0.54 | 0.62716 |
| | 107 | | | | | |
| Gpr108 | G protein-coupled receptor | 8.36 | 0.39 | 8.12 | 0.38 | 0.39528 |
| | 108 | | | | | |
| Gpr110 | G protein-coupled receptor | 7.71 | 1.96 | 3.83 | 0.95 | 0.00001 |
| | 110 | | | | | |
| Gpr113 | G protein-coupled receptor | 6.84 | 0.31 | 7.36 | 0.38 | 0.04368 |
| | 113 | | | | | |
| Gpr115 | G protein-coupled receptor | 4.6 | 0.30 | 4.79 | 0.29 | 0.67355 |
| | 115 | | | | | |
| Gpr120 | G protein-coupled receptor | 5.58 | 0.38 | 5.77 | 0.38 | 0.64680 |
| | 120 | | | | | |
| Gpr123 | G protein-coupled receptor | 8.27 | 0.35 | 8.33 | 0.34 | 0.84204 |
| | 123 | | | | | |
| Gpr124 | G protein-coupled receptor | 5.04 | 1.40 | 5.49 | 1.38 | 0.53999 |
| | 124 | | | | | |
| Gpr126 | G protein-coupled receptor | 4.97 | 0.87 | 5.2 | 0.94 | 0.63814 |
| | 126 | | | | | |
| Gpr128 | G protein-coupled receptor | 5.7 | 0.58 | 5.54 | 0.59 | 0.73760 |
| | 128 | | | | | |
| Gpr132 | G protein-coupled receptor | 4.76 | 0.36 | 4.8 | 0.39 | 0.88371 |
| | 132 | | | | | |
| Gpr135 | G protein-coupled receptor | 6 | 0.23 | 6.25 | 0.21 | 0.07325 |
| | 135 | | | | | |
| Gpr137 | G protein-coupled receptor | 6.71 | 0.55 | 6.82 | 0.55 | 0.77283 |
| | 137 | | | | | |
| Gpr137b- | G protein-coupled receptor | 6.07 | 0.48 | 6.55 | 0.55 | 0.20238 |
| ps | 137B, pseudogene | | | | | |
| Gpr137c | G protein-coupled receptor | 7.3 | 0.53 | 7.51 | 0.51 | 0.42757 |
| | 137C | | | | | |
| Gpr141 | G protein-coupled receptor | 3.35 | 1.47 | 2.73 | 1.36 | 0.38175 |
| | 141 | | | | | |
| Gpr142 | G protein-coupled receptor | 4.28 | 0.54 | 4.23 | 0.57 | 0.92814 |
| | 142 | | | | | |
| Gpr143 | G protein-coupled receptor | 7.46 | 0.55 | 7.76 | 0.48 | 0.24913 |
| | 143 | | | | | |
| Gpr146 | G protein-coupled receptor | 9.92 | 0.35 | 9.62 | 0.32 | 0.10740 |
| | 146 | | | | | |
| Gpr149 | G protein-coupled receptor | 2.46 | 1.77 | 3.15 | 1.42 | 0.46170 |
| | 149 | | | | | |
| Gpr150 | G protein-coupled receptor | 3.6 | 1.77 | 4.59 | 0.50 | 0.23267 |
| | 150 | | | | | |
| Gpr151 | G protein-coupled receptor | 6.17 | 0.33 | 6.45 | 0.42 | 0.38221 |
| | 151 | | | | | |
| Gpr152 | G protein-coupled receptor | 5.48 | 0.55 | 5.14 | 0.43 | 0.24115 |
| | 152 | | | | | |
| Gpr153 | G protein-coupled receptor | 7.72 | 0.36 | 7.88 | 0.33 | 0.54607 |
| | 153 | | | | | |
| Gpr156 | G protein-coupled receptor | 6 | 0.43 | 6.24 | 0.42 | 0.40792 |
| | 156 | | | | | |
| Gpr157 | G protein-coupled receptor | 7.22 | 0.20 | 7.47 | 0.21 | 0.15631 |
| | 157 | | | | | |
| Gpr158 | G protein-coupled receptor | 5.97 | 0.45 | 6.21 | 0.45 | 0.38980 |
| | 158 | | | | | |
| Gpr162 | G protein-coupled receptor | 4.12 | 1.30 | 4.31 | 1.29 | 0.79321 |
| | 162 | | | | | |
| Gpr17 | G protein-coupled receptor | 5.71 | 0.39 | 5.7 | 0.37 | 0.97438 |
| | 17 | | | | | |
| Gpr171 | G protein-coupled receptor | 3.74 | 1.83 | 3.7 | 1.69 | 0.95894 |
| | 171 | | | | | |
| Gpr172b | G protein-coupled receptor | 6.6 | 0.61 | 6.53 | 0.63 | 0.87711 |
| | 172B | | | | | |
| Gpr176 | G protein-coupled receptor | 4.09 | 0.62 | 4.63 | 0.61 | 0.23916 |
| | 176 | | | | | |
| Gpr18 | G protein-coupled receptor | 3.55 | 1.46 | 3.27 | 1.48 | 0.69476 |
| | 18 | | | | | |
| Gpr180 | G protein-coupled receptor | 8.69 | 0.32 | 8.64 | 0.31 | 0.79329 |
| | 180 | | | | | |
| Gpr182 | G protein-coupled receptor | 8.48 | 0.41 | 8.7 | 0.41 | 0.31468 |
| | 182 | | | | | |
| Gpr183 | G protein-coupled receptor | 6.61 | 0.37 | 6.46 | 0.32 | 0.56357 |
| | 183 | | | | | |
| Gpr19 | G protein-coupled receptor | 5.28 | 0.60 | 5.53 | 0.58 | 0.60395 |
| | 19 | | | | | |
| Gpr20 | G protein-coupled receptor | 5.38 | 0.48 | 5.47 | 0.44 | 0.74193 |
| | 20 | | | | | |
| Gpr21 | G protein-coupled receptor | 3.29 | 1.00 | 3.68 | 1.05 | 0.59572 |
| | 21 | | | | | |
| Gpr22 | G protein-coupled receptor | 4.87 | 0.40 | 4.55 | 0.33 | 0.14974 |
| | 22 | | | | | |
| Gpr26 | G protein-coupled receptor | 7.08 | 0.28 | 7.07 | 0.28 | 0.94997 |
| | 26 | | | | | |
| Gpr27 | G protein-coupled receptor | 6.94 | 0.37 | 6.96 | 0.34 | 0.92842 |
| | 27 | | | | | |
| Gpr30 | G protein-coupled receptor | 5.81 | 0.40 | 5.95 | 0.27 | 0.60764 |
| | 30 | | | | | |
| Gpr33 | G protein-coupled receptor | 4.37 | 0.24 | 4.15 | 0.22 | 0.15585 |
| | 33 | | | | | |
| Gpr34 | G protein-coupled receptor | 3.41 | 0.79 | 3.48 | 0.75 | 0.86326 |
| | 34 | | | | | |
| Gpr35 | G protein-coupled receptor | 5.49 | 0.40 | 5.94 | 0.42 | 0.17594 |
| | 35 | | | | | |
| Gpr37 | G protein-coupled receptor | 5.57 | 0.40 | 5.97 | 0.36 | 0.25086 |
| | 37 | | | | | |
| Gpr37l1 | G protein-coupled receptor | 5.08 | 0.44 | 5.51 | 0.46 | 0.23055 |
| | 37-like 1 | | | | | |
| Gpr39 | G protein-coupled receptor | 6.73 | 0.59 | 6.76 | 0.59 | 0.94250 |
| | 39 | | | | | |
| Gpr4 | G protein-coupled receptor 4 | 4.67 | 0.45 | 4.9 | 0.41 | 0.42856 |
| Gpr44 | G protein-coupled receptor | 5.87 | 0.36 | 6.12 | 0.27 | 0.45746 |
| | 44 | | | | | |
| Gpr45 | G protein-coupled receptor | 4.35 | 0.65 | 4.51 | 0.68 | 0.75453 |
| | 45 | | | | | |
| Gpr56 | G protein-coupled receptor | 6.58 | 0.32 | 6.78 | 0.32 | 0.60614 |
| | 56 | | | | | |
| Gpr6 | G protein-coupled receptor 6 | 4.25 | 0.81 | 4.21 | 0.84 | 0.92822 |
| Gpr61 | G protein-coupled receptor | 5.61 | 0.37 | 5.77 | 0.41 | 0.60697 |
| | 61 | | | | | |
| Gpr63 | G protein-coupled receptor | 4.55 | 0.35 | 4.59 | 0.34 | 0.91106 |
| | 63 | | | | | |
| Gpr64 | G protein-coupled receptor | 4.3 | 1.20 | 5.69 | 1.16 | 0.08922 |
| | 64 | | | | | |
| Gpr68 | G protein-coupled receptor | 6.16 | 0.34 | 6.16 | 0.34 | 0.99817 |
| | 68 | | | | | |
| Gpr75 | G protein-coupled receptor | 5.49 | 0.36 | 5.51 | 0.41 | 0.94538 |
| | 75 | | | | | |
| Gpr77 | G protein-coupled receptor | 5.67 | 0.35 | 6.11 | 0.36 | 0.08388 |
| | 77 | | | | | |
| Gpr81 | G protein-coupled receptor | 4.93 | 0.49 | 5.53 | 0.28 | 0.16911 |
| | 81 | | | | | |
| Gpr82 | G protein-coupled receptor | 3.63 | 0.44 | 3.45 | 0.39 | 0.66910 |
| | 82 | | | | | |
| Gpr83 | G protein-coupled receptor | 4.2 | 0.34 | 4.88 | 0.42 | 0.05833 |
| | 83 | | | | | |
| Gpr84 | G protein-coupled receptor | 3.52 | 0.29 | 4.6 | 0.33 | 0.16888 |
| | 84 | | | | | |
| Gpr85 | G protein-coupled receptor | 7.46 | 0.40 | 7.63 | 0.39 | 0.57426 |
| | 85 | | | | | |
| Gpr87 | G protein-coupled receptor | 5.91 | 0.82 | 5.85 | 0.81 | 0.91283 |
| | 87 | | | | | |
| Gpr89 | G protein-coupled receptor | 8.38 | 0.49 | 8.29 | 0.46 | 0.76141 |
| | 89 | | | | | |
| Gpr97 | G protein-coupled receptor | 6.14 | 0.37 | 6.59 | 0.19 | 0.10953 |
| | 97 | | | | | |
| Gpr98 | G protein-coupled receptor | 6.17 | 0.67 | 6.97 | 0.47 | 0.01870 |
| | 98 | | | | | |
| Gprc5a | G protein-coupled receptor, | 3.95 | 0.73 | 4.03 | 0.71 | 0.88108 |
| | family C, group 5, member A | | | | | |
| Gprc5b | G protein-coupled receptor, | 2.5 | 1.86 | 4.5 | 1.85 | 0.06886 |
| | family C, group 5, member B | | | | | |
| Gprc5c | G protein-coupled receptor, | 8.5 | 0.33 | 8.47 | 0.38 | 0.90230 |
| | family C, group 5, member C | | | | | |
| Gprc5c | G protein-coupled receptor, | 7.78 | 0.46 | 7.85 | 0.52 | 0.82192 |
| | family C, group 5, member C | | | | | |
| Gprc5d | G protein-coupled receptor, | 3.23 | 1.15 | 3.31 | 1.19 | 0.92889 |
| | family C, group 5, member D | | | | | |
| Gprc6a | G protein-coupled receptor, | 5.06 | 0.75 | 5.78 | 0.48 | 0.14073 |
| | family C, group 6, member A | | | | | |
| Galr2 | galanin receptor 2 | 5.66 | 0.28 | 5.62 | 0.29 | 0.86159 |
| Galr3 | galanin receptor 3 | 5.92 | 0.35 | 6.28 | 0.24 | 0.36657 |
| Grpr | gastrin releasing peptide | 2.85 | 0.56 | 3.89 | 0.60 | 0.12143 |
| | receptor | | | | | |
| Gnrhr | gonadotropin releasing | 4.36 | 0.43 | 4.47 | 0.32 | 0.74652 |
| | hormone receptor | | | | | |
| Ghsr | growth hormone | 6.31 | 0.30 | 6.41 | 0.31 | 0.59179 |
| | secretagogue receptor | | | | | |
| Hrh1 | histamine receptor H1 | 4.98 | 0.34 | 5.21 | 0.31 | 0.19105 |
| Hrh2 | histamine receptor H2 | 4.18 | 0.92 | 2.91 | 0.78 | 0.00692 |
| Hrh3 | histamine receptor H3 | 7.97 | 0.44 | 8.14 | 0.38 | 0.46158 |
| Hrh4 | histamine receptor H4 | 4 | 0.48 | 3.77 | 0.47 | 0.41377 |
| Hcrtr1 | hypocretin (orexin) receptor 1 | 5.15 | 0.63 | 5.79 | 0.47 | 0.16738 |
| Hcrtr2 | hypocretin (orexin) receptor 2 | 5.43 | 0.94 | 5.13 | 0.91 | 0.51489 |
| Kiss1r | KISS1 receptor | 6.12 | 0.82 | 6.81 | 0.66 | 0.23507 |
| Lgr5 | leucine rich repeat containing | 7.03 | 0.60 | 7.78 | 0.59 | 0.10732 |
| | G protein coupled receptor 5 | | | | | |
| Ltb4r1 | leukotriene B4 receptor 1 | 5.94 | 0.16 | 5.91 | 0.15 | 0.89357 |
| Ltb4r2 | leukotriene B4 receptor 2 | 6.56 | 0.44 | 6.8 | 0.33 | 0.31610 |
| Lhcgr | luteinizing | 5.56 | 0.51 | 5.33 | 0.51 | 0.49212 |
| | hormone/choriogonadotropin | | | | | |
| | receptor | | | | | |
| Lpar1 | lysophosphatidic acid | 6.56 | 0.34 | 6.81 | 0.33 | 0.33956 |
| | receptor 1 | | | | | |
| Lpar2 | lysophosphatidic acid | 5.55 | 0.35 | 6.11 | 0.20 | 0.12832 |
| | receptor 2 | | | | | |
| Lpar3 | lysophosphatidic acid | 8.52 | 0.31 | 8.81 | 0.30 | 0.12775 |
| | receptor 3 | | | | | |
| Lpar4 | lysophosphatidic acid | 4.49 | 0.37 | 4.46 | 0.35 | 0.89175 |
| | receptor 4 | | | | | |
| Lpar6 | lysophosphatidic acid | 8.99 | 0.31 | 9.36 | 0.32 | 0.17935 |
| | receptor 6 | | | | | |
| Mas1 | MAS1 oncogene | 4.67 | 0.72 | 5.25 | 0.73 | 0.17770 |
| Mrgprd | MAS-related GPR, member | 4.74 | 0.48 | 5.3 | 0.29 | 0.10883 |
| | D | | | | | |
| Mrgpre | MAS-related GPR, member E | 6.22 | 0.38 | 6.39 | 0.37 | 0.40279 |
| Mrgprf | MAS-related GPR, member F | 6.13 | 0.99 | 6.46 | 1.02 | 0.60085 |
| Mrgprg | MAS-related GPR, member | 5.52 | 0.36 | 5.42 | 0.35 | 0.66871 |
| | G | | | | | |
| Mrgprx1 | MAS-related GPR, member | 3.19 | 0.16 | 2.4 | 0.17 | 0.02077 |
| | X1 | | | | | |
| Mchr1 | melanin-concentrating | 3.26 | 1.32 | 3.01 | 1.41 | 0.75052 |
| | hormone receptor 1 | | | | | |
| Mc1r | melanocortin 1 receptor | 4.62 | 0.29 | 4.7 | 0.32 | 0.78990 |
| Mc2r | melanocortin 2 receptor | 4.41 | 0.29 | 4.65 | 0.36 | 0.26810 |
| Mc3r | melanocortin 3 receptor | 3.77 | 0.35 | 3.77 | 0.43 | 0.99588 |
| Mc4r | melanocortin 4 receptor | 5.09 | 0.58 | 5.17 | 0.51 | 0.82331 |
| Mc5r | melanocortin 5 receptor | 5.1 | 0.19 | 5.06 | 0.18 | 0.89032 |
| Mtnr1a | melatonin receptor 1A | 6.25 | 0.56 | 7.62 | 0.53 | 0.01514 |
| Mtnr1b | melatonin receptor 1B | 4.49 | 1.11 | 3.95 | 1.17 | 0.34537 |
| Nmbr | neuromedin B receptor | 4.8 | 0.25 | 5.09 | 0.27 | 0.09007 |
| Nmur1 | neuromedin U receptor 1 | 4.19 | 0.94 | 3.05 | 1.05 | 0.06309 |
| Nmur2 | neuromedin U receptor 2 | 4.51 | 0.55 | 4.35 | 0.59 | 0.65706 |
| Npffr2 | neuropeptide FF receptor 2 | 5.76 | 0.36 | 5.46 | 0.40 | 0.33905 |
| Npsr1 | neuropeptide S receptor 1 | 5.25 | 0.53 | 5.7 | 0.52 | 0.31063 |
| Npy1r | neuropeptide Y receptor Y1 | 4 | 0.54 | 4.57 | 0.50 | 0.05136 |
| Npy2r | neuropeptide Y receptor Y2 | 3.85 | 0.73 | 4.41 | 0.75 | 0.53172 |
| Npy5r | neuropeptide Y receptor Y5 | 4.6 | 0.41 | 4.34 | 0.29 | 0.30368 |
| Npy6r | neuropeptide Y receptor Y6 | 3.02 | 0.43 | 3.47 | 0.34 | 0.22168 |
| Ntsr1 | neurotensin receptor 1 | 6.92 | 0.51 | 7.03 | 0.50 | 0.79865 |
| Ntsr2 | neurotensin receptor 2 | 8.69 | 0.25 | 8.95 | 0.27 | 0.30427 |
| Oprd1 | opioid receptor, delta 1 | 6.91 | 0.43 | 7.03 | 0.38 | 0.67278 |
| Oprk1 | opioid receptor, kappa 1 | 3.78 | 0.57 | 3.79 | 0.54 | 0.97964 |
| Oprm1 | opioid receptor, mu 1 | 5.98 | 0.47 | 6.13 | 0.46 | 0.67908 |
| Oprl1 | opioid receptor-like 1 | 5 | 0.19 | 5.31 | 0.18 | 0.20871 |
| Opn1mw | opsin 1 (cone pigments), | 3.76 | 0.90 | 3.93 | 0.95 | 0.84814 |
| | medium-wave-sensitive | | | | | |
| | (color blindness, deutan) | | | | | |
| Opn1sw | opsin 1 (cone pigments), | 5.52 | 0.57 | 5.65 | 0.59 | 0.73438 |
| | short-wave-sensitive (color | | | | | |
| | blindness, tritan) | | | | | |
| Opn3 | opsin 3 | 5.89 | 0.40 | 5.16 | 0.26 | 0.00411 |
| Opn4 | opsin 4 (melanopsin) | 6.81 | 0.41 | 7.19 | 0.34 | 0.24053 |
| Opn5 | opsin 5 | 4.88 | 0.45 | 5.34 | 0.44 | 0.14551 |
| Oxgr1 | oxoglutarate (alpha- | 3.32 | 0.35 | 3.51 | 0.38 | 0.59082 |
| | ketoglutarate) receptor 1 | | | | | |
| Oxtr | oxytocin receptor | 4.99 | 0.87 | 5.26 | 0.58 | 0.54141 |
| Prokr1 | prokineticin receptor 1 | 3.67 | 0.48 | 4.18 | 0.43 | 0.50837 |
| Prokr2 | prokineticin receptor 2 | 6.45 | 0.29 | 6.41 | 0.28 | 0.83847 |
| Prlhr | prolactin releasing hormone | 3.58 | 0.37 | 3.45 | 0.37 | 0.62331 |
| | receptor | | | | | |
| Ptgdr | prostaglandin D receptor | 4.02 | 0.51 | 3.72 | 0.59 | 0.34160 |
| Ptger1 | prostaglandin E receptor 1 | 4.66 | 0.56 | 4.5 | 0.47 | 0.63530 |
| | (subtype EP1) | | | | | |
| Ptger2 | prostaglandin E receptor 2 | 4.65 | 0.26 | 5.29 | 0.16 | 0.03072 |
| | (subtype EP2) | | | | | |
| Ptger3 | prostaglandin E receptor 3 | 3.92 | 0.42 | 4.95 | 0.37 | 0.23612 |
| | (subtype EP3) | | | | | |
| Ptger4 | prostaglandin E receptor 4 | 4.74 | 0.30 | 5.22 | 0.29 | 0.22462 |
| | (subtype EP4) | | | | | |
| Ptgfr | prostaglandin F receptor | 3.87 | 0.55 | 4.76 | 0.39 | 0.03679 |
| Ptgir | prostaglandin I receptor (IP) | 5.29 | 0.52 | 5.24 | 0.51 | 0.86514 |
| P2ry1 | purinergic receptor P2Y, G- | 8.36 | 0.43 | 8.83 | 0.33 | 0.10199 |
| | protein coupled 1 | | | | | |
| P2ry10 | purinergic receptor P2Y, G- | 2.57 | 0.85 | 3.44 | 0.83 | 0.15845 |
| | protein coupled 10 | | | | | |
| P2ry12 | purinergic receptor P2Y, G- | 6.34 | 0.18 | 6.59 | 0.28 | 0.34222 |
| | protein coupled 12 | | | | | |
| P2ry13 | purinergic receptor P2Y, G- | 6.68 | 0.21 | 6.74 | 0.29 | 0.71166 |
| | protein coupled 13 | | | | | |
| P2ry2 | purinergic receptor P2Y, G- | 8.83 | 0.59 | 8.79 | 0.62 | 0.91505 |
| | protein coupled 2 | | | | | |
| P2ry14 | purinergic receptor P2Y, G- | 6.01 | 0.56 | 6.68 | 0.68 | 0.10995 |
| | protein coupled, 14 | | | | | |
| Qrfpr | pyroglutamylated RFamide | 6.62 | 0.37 | 6.14 | 0.30 | 0.00943 |
| | peptide receptor | | | | | |
| Rxfp3 | relaxin family peptide | 4.08 | 1.52 | 3.16 | 1.68 | 0.27344 |
| | receptor 3 | | | | | |
| Rxfp4 | relaxin family peptide | 3.54 | 0.90 | 4.81 | 0.50 | 0.01682 |
| | receptor 4 | | | | | |
| Rxfp1 | relaxin/insulin-like family | 5.26 | 0.49 | 5.38 | 0.51 | 0.76588 |
| | peptide receptor 1 | | | | | |
| Rxfp2 | relaxin/insulin-like family | 5.73 | 0.47 | 5.2 | 0.38 | 0.05139 |
| | peptide receptor 2 | | | | | |
| Sstr1 | somatostatin receptor 1 | 7.41 | 0.39 | 7.6 | 0.32 | 0.54433 |
| Sstr3 | somatostatin receptor 3 | 6.56 | 0.11 | 6.56 | 0.12 | 0.99291 |
| Sstr4 | somatostatin receptor 4 | 7.12 | 0.32 | 7.06 | 0.34 | 0.76321 |
| S1pr1 | sphingosine-1-phosphate | 8.84 | 0.34 | 9.4 | 0.33 | 0.05902 |
| | receptor 1 | | | | | |
| S1pr2 | sphingosine-1-phosphate | 5.59 | 0.63 | 5.81 | 0.62 | 0.60675 |
| | receptor 2 | | | | | |
| S1pr3 | sphingosine-1-phosphate | 6.02 | 0.44 | 6.31 | 0.49 | 0.41398 |
| | receptor 3 | | | | | |
| S1pr4 | sphingosine-1-phosphate | 4.57 | 0.65 | 4.28 | 0.70 | 0.44313 |
| | receptor 4 | | | | | |
| S1pr5 | sphingosine-1-phosphate | 6.7 | 0.31 | 6.82 | 0.49 | 0.72330 |
| | receptor 5 | | | | | |
| Sucnr1 | succinate receptor 1 | 8.82 | 0.25 | 9.39 | 0.29 | 0.09572 |
| Tacr1 | tachykinin receptor 1 | 4.95 | 0.51 | 5.81 | 0.26 | 0.15301 |
| Tacr2 | tachykinin receptor 2 | 5.59 | 0.68 | 5.74 | 0.66 | 0.77054 |
| Tacr3 | tachykinin receptor 3 | 4.82 | 0.24 | 5.49 | 0.23 | 0.26982 |
| Tbxa2r | thromboxane A2 receptor | 4.57 | 0.80 | 5.83 | 0.60 | 0.23528 |
| Tshr | thyroid stimulating hormone | 5.12 | 0.44 | 5.44 | 0.47 | 0.23447 |
| | receptor | | | | | |
| Trhr | thyrotropin releasing | 4.94 | 0.46 | 4.66 | 0.38 | 0.32957 |
| | hormone receptor | | | | | |
| Trhr2 | thyrotropin releasing | 5.39 | 0.53 | 5.39 | 0.52 | 0.99232 |
| | hormone receptor 2 | | | | | |
| Taar1 | trace amine-associated | 4.86 | 0.25 | 5.15 | 0.25 | 0.23825 |
| | receptor 1 | | | | | |
| Taar2 | trace amine-associated | 1.76 | 0.85 | 2.3 | 0.88 | 0.36888 |
| | receptor 2 | | | | | |
| Taar3 | trace amine-associated | 3.39 | 0.65 | 3.37 | 0.64 | 0.96888 |
| | receptor 3 | | | | | |
| Taar4 | trace amine-associated | 4.17 | 0.38 | 4.49 | 0.37 | 0.31870 |
| | receptor 4 | | | | | |
| Taar5 | trace amine-associated | 5.19 | 0.21 | 5.47 | 0.07 | 0.12264 |
| | receptor 5 | | | | | |
| Taar6 | trace amine-associated | 7.27 | 0.38 | 7.37 | 0.37 | 0.69455 |
| | receptor 6 | | | | | |
| Taar7a | trace amine-associated | 0.71 | 0.94 | 1.03 | 1.00 | 0.56784 |
| | receptor 7A | | | | | |
| Taar7b// | trace amine-associated | 5.6 | 0.63 | 6.36 | 0.62 | 0.19868 |
| Taar7b | receptor 7B//trace amine- | | | | | |
| | associated receptor 7B | | | | | |
| Taar7d | trace amine-associated | 2.06 | 1.01 | 2.52 | 0.85 | 0.52164 |
| | receptor 7D | | | | | |
| Taar7e | trace amine-associated | 1.43 | 1.40 | 1.24 | 1.34 | 0.84692 |
| | receptor 7E | | | | | |
| Taar7f | trace amine-associated | 1.97 | 1.12 | 1.92 | 1.40 | 0.94460 |
| | receptor 7F | | | | | |
| Taar8b | trace amine-associated | 0.42 | 0.68 | 1.16 | 0.62 | 0.10033 |
| | receptor 8B | | | | | |
| Taar9 | trace amine-associated | 4.58 | 0.23 | 4.7 | 0.22 | 0.46412 |
| | receptor 9 | | | | | |
| Uts2r | urotensin 2 receptor | 6.85 | 0.56 | 6.9 | 0.56 | 0.90845 |

Typically, for microarray analysis, the liver of mice fed with either STC (n=6) or HFD (n=6) for 8 weeks were sent for gene expression analysis using Affymetrix Mouse Exon 1.0 ST Array. RNA was extracted from the liver of mice treated with rAAV-GPR110 and ASO-GPR110 using RNeasy Kits (QIAGEN, Hilden, Germany). RNA concentration was quantified using NanoDrop™ 2000 Spectrophotometer (Thermo Scientific, Waltham, USA) and RNA quality was assessed using Agilent 2100 Bioanalyzer (Agilent, Santa Clara, USA). 10 μg of total RNA from liver with RNA integrity number (RIN) greater than 7 was used in RNA-seq. RNA-seq was performed by BGI and analyzed by Dr. Tom system (BGI, Shenzhen, China).

Table 2 below provides expression of lipogenic genes in the liver of mice fed with either STC or HFD diet for 8 weeks by gene expression microarray analysis.

TABLE 2

| Gene | | STC | | HFD | | p |
|---|---|---|---|---|---|---|
| Symbol | Gene Description | average | SD | average | SD | value |
| SCD1 | stearoyl-Coenzyme A desaturase 1 | 12.61 | 0.64 | 13.36 | 0.22 | 0.02 |
| FASN | fatty acid synthase | 9.22 | 0.43 | 10.04 | 1.81 | 0.05 |
| Acacb | acetyl-Coenzyme A carboxylase | 8.62 | 0.91 | 8.99 | 0.98 | 0.41 |
| | beta | | | | | |
| Srebf1 | sterol regulatory element binding | 9.07 | 0.59 | 10.21 | 0.59 | 0.00 |
| | transcription | | | | | |
| | factor 1 | | | | | |
| Ppara | peroxisome proliferator activated | 11.23 | 0.53 | 11.48 | 0.82 | 0.21 |
| | receptor | | | | | |
| | alpha | | | | | |
| Pparg | peroxisome proliferator activated | 7.49 | 0.31 | 8.88 | 1.63 | 0.01 |
| | receptor | | | | | |
| | gamma | | | | | |
| Ppard | peroxisome proliferator activator | 6.31 | 0.89 | 6.68 | 1.29 | 0.28 |
| | receptor delta | | | | | |
| Usf1 | upstream transcription factor 1 | 7.02 | 0.59 | 6.74 | 0.31 | 0.33 |
| Acly | ATP citrate lyase | 10.44 | 0.43 | 10.54 | 0.49 | 0.71 |
| Accs | 1-aminocyclopropane-1-carboxylate | 3.68 | 1.84 | 5.14 | 0.67 | 0.10 |
| | synthase | | | | | |
| | homolog (Arabidopsis)(non- | | | | | |
| | functional) | | | | | |
| Hdac3 | histone deacetylase 3 | 9.75 | 0.29 | 10.19 | 0.17 | 0.01 |
| Ncor1 | nuclear receptor co-repressor 1 | 9.24 | 0.79 | 9.70 | 0.79 | 0.34 |
| Ncor2 | nuclear receptor co-repressor 2 | 6.78 | 0.89 | 7.28 | 0.93 | 0.36 |
| Hdac9 | histone deacetylase 9 | 6.87 | 0.54 | 7.55 | 0.74 | 0.10 |
| Usf2 | upstream transcription factor 2 | 9.44 | 0.43 | 9.52 | 0.29 | 0.72 |
| Usf1 | upstream transcription factor 1 | 7.02 | 0.59 | 6.74 | 0.31 | 0.33 |
| Mtor | mechanistic target of rapamycin | 8.18 | 0.72 | 8.55 | 0.74 | 0.41 |
| | (serine/threonine kinase) | | | | | |
| Akt1 | thymoma viral proto-oncogene 1 | 9.24 | 0.48 | 9.26 | 0.55 | 0.95 |
| Akt3 | thymoma viral proto-oncogene 3 | 7.10 | 0.57 | 7.76 | 0.51 | 0.06 |
| Tsc2 | tuberous sclerosis 2 | 7.91 | 0.74 | 8.50 | 0.83 | 0.23 |
| Tsc1 | tuberous sclerosis 1 | 6.98 | 0.81 | 7.23 | 0.89 | 0.62 |
| Ncoa6 | nuclear receptor coactivator 6 | 7.26 | 1.30 | 7.80 | 1.38 | 0.50 |
| Rxra | retinoid X receptor alpha | 10.39 | 0.81 | 10.99 | 0.74 | 0.21 |
| Rxrb | retinoid X receptor beta | 7.18 | 0.49 | 7.07 | 0.42 | 0.68 |
| Rxrg | retinoid X receptor gamma | 7.83 | 0.27 | 8.45 | 0.41 | 0.01 |

Next, cell fractionation was used to identify the GPR110 expressing cells in liver. CD11b mRNAs were used as markers for non-parenchymal cells (NPCs), and albumin mRNA for hepatocytes. The cell fractionation clearly demonstrated that GPR110 mRNA is mainly expressed in hepatocytes (FIG. 1C). This finding was further supported by Western blot analysis (FIG. 1D). In agreement with the microarray data, during HFD treatment for 8 weeks, the expression level of hepatic GPR110 gradually declined and to almost undetectable level at week 8 as examined by RT-qPCR analysis (FIG. 1E). Typically, total RNA was extracted with RNAiso Plus (#9109, TakaRa Bio Inc., Shiga, Japan). RNA was then reverse transcribed into cDNA with PRIMESCRIPT™ RT reagent Kit (#RR037, TakaRa Bio Inc., Shiga, Japan). cDNA was then amplified with TB green Premix Ex Taq™ II (Til RNase). The real-time PCR was conducted with a LightCycler 96 RT-qPCR System (Roche, Basel, Switzerland). The relative quantity of the targeted RNA was calculated through normalization to the quantity of the corresponding GAPDH mRNA level. Table 3 provides primers used for RT-qPCR:

TABLE 3

| Gene | Primer sequences (5′-3′) | | SEQ ID |
|---|---|---|---|
| name | Forward | Reverse | No |
| GPR110 | CCAAGAGAAGCCAAACCTCC | TTCGATAAGCCAGCAGGATG | 3 & 4 |
| SCD1 | CTGACCTGAAAGCCGAGAAG | AGAAGGTGCTAACGAACAGG | 5 & 6 |
| GAPDH | ACTCCACTCACGGCAAATTC | TCTCCATGGTGGTGAAGACA | 7 & 8 |
| Albumin | ACAGGACACCTGCTCTC | AGTCCTGAGTCCTTCATGTCT | 9 & 10 |
| | | TT | |
| F4/80 | CTTTGGCTATGGGCCTTCCAG | GCAAGGAGGACAGAGTTTAT | 11 & 12 |
| | TC | CGTG | |
| CD11b | ATGGACGCTGATGGCAATAC | TCCCCATTCACGTCTCCCA | 13 & 14 |
| | C | | |
| Acot1 | ACTACGATGACCTCCCCAAG | CATAGCAAGGCCAAGTTCAC | 15 & 16 |
| Cy4a12b | GTTCCTACAGATTTCTAGCTC | AGAGTCTGCCATGATTTCCG | 17 & 18 |
| | CC | | |
| Cy4a31 | CACTCATTCCTGCCCTTCTC | ACAATCACCTTCAGCTCACTC | 19 & 20 |
| Acaca | AAGGCTATGTGAAGGATGTG | CTGTCTGAAGAGGTTAGGGA | 21 & 22 |
| | G | AG | |
| Pcsk9 | TTTTATGACCTCTTCCCTGGC | ATTCGCTCCAGGTTCCATG | 23 & 24 |
| Mrp153 | TCAAGCTGGTTCGAGTTCAG | ACAGAGCAGTTGAGGTTGG | 25 & 26 |
| Hspd1 | AGTGTTCAGTCCATTGTCCC | TGACTGCCACAACCTGAAG | 27 & 28 |
| Pltp | CCTGTGCTCTACCATGCTG | ATTCCATATCCAGGTTGCCG | 29 & 30 |
| Abca1 | TGACATGGTACATCGAAGCC | GATTTCTGACACTCCCTTCTG | 31 & 32 |
| | | G | |
| FGF21 | ACGACCAAGACACTGAAGC | ACCCAGGATTTGAATGACCC | 33 & 34 |

In contrast, the mRNA levels of the NAFLD related marker FGF21 were highly induced in the livers of HFD fed mice at week 8 (FIG. 1F). Western blot analysis was performed to confirm that the declined expression of GPR110 is also observed in protein level in the livers of HFD-fed mice (FIG. 1G, left panel). Interestingly, HFD-treatment did not affect the renal GRP110 expression (FIG. 1G, right panel). Collectively, the hepatic, but not renal, GPR110 level is tightly regulated by nutritional status.

### Example 2—Overexpression of GPR110 in Hepatocytes Accelerates Metabolic Dysregulation Caused by HFD

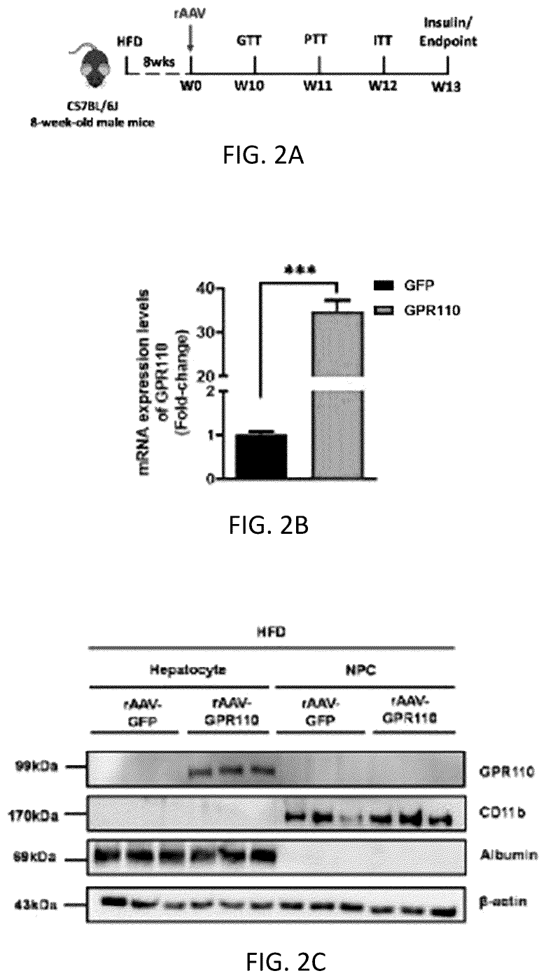

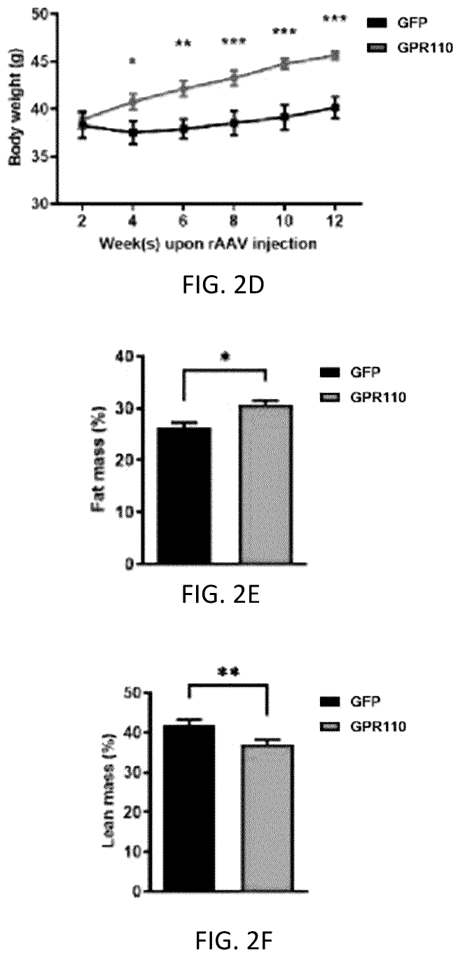

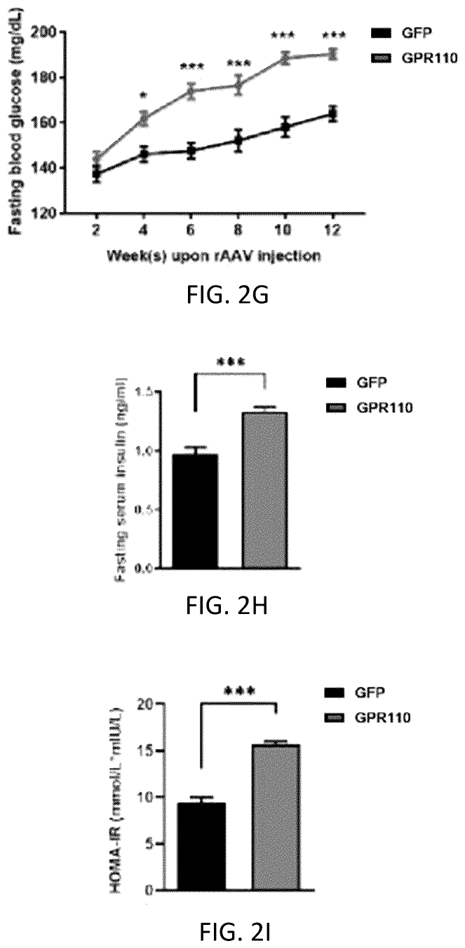

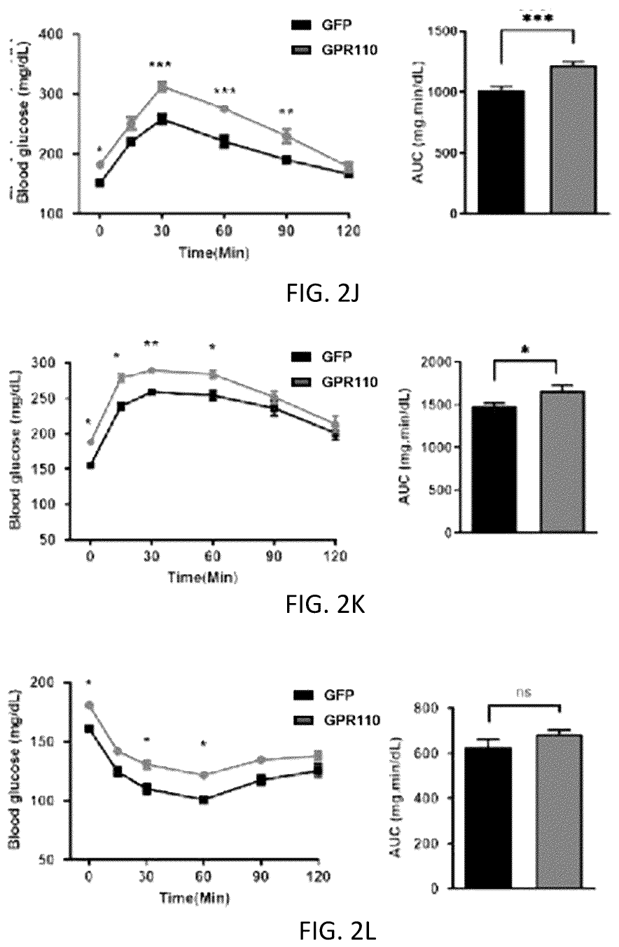

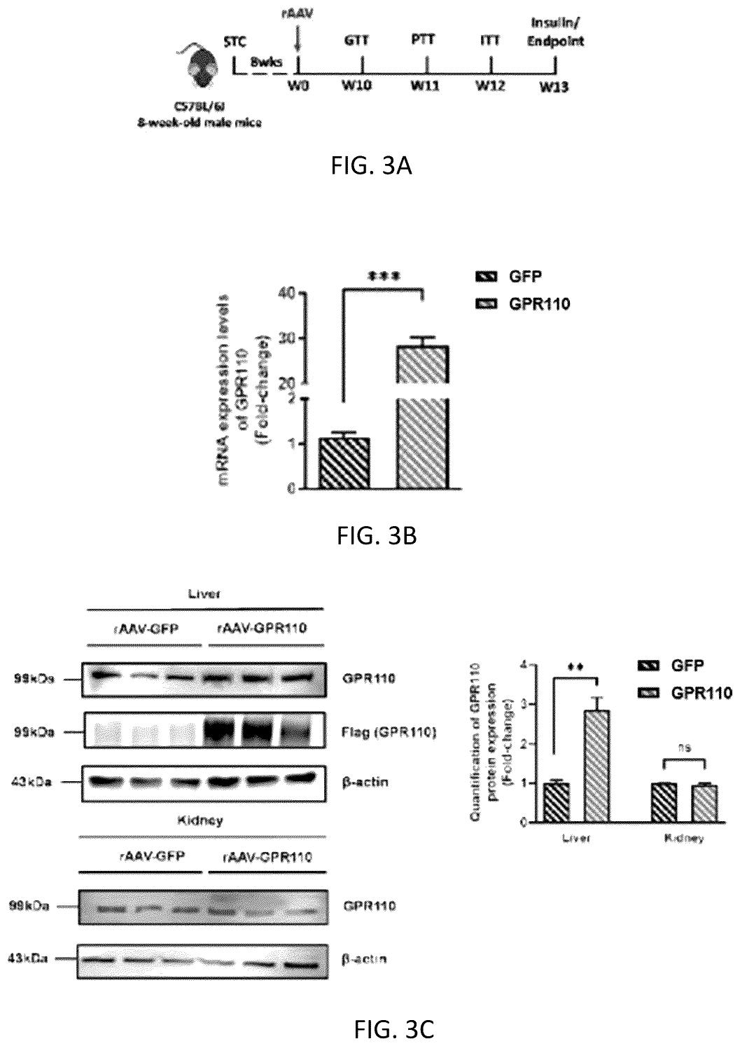

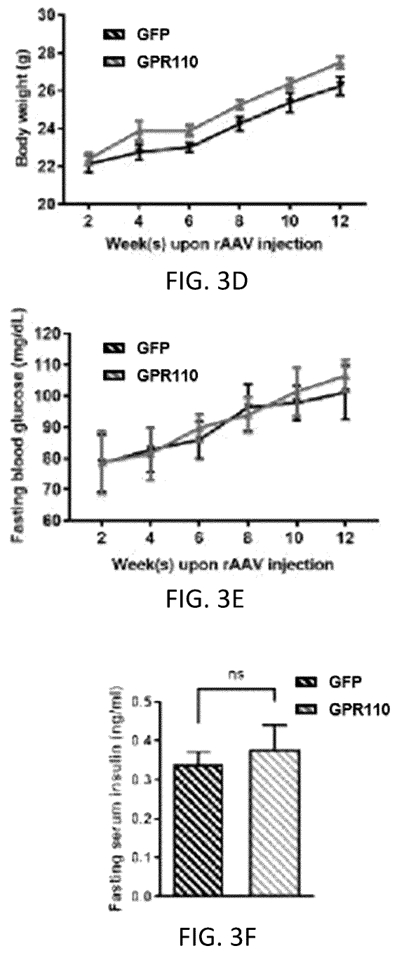

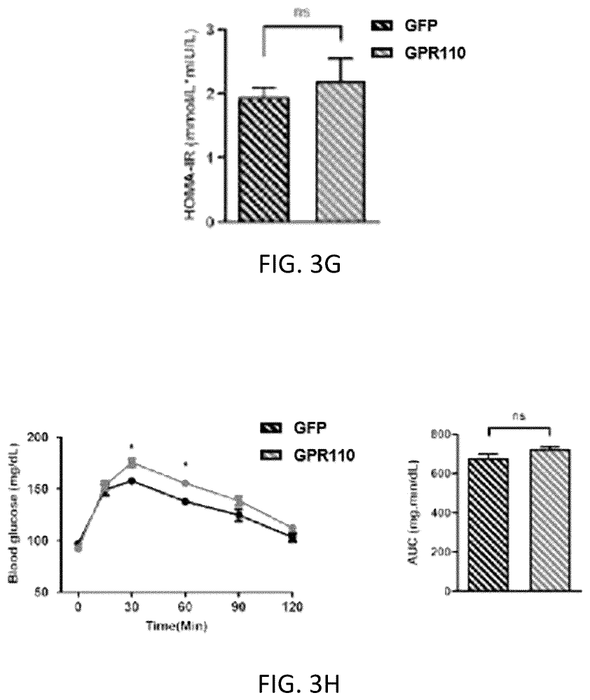

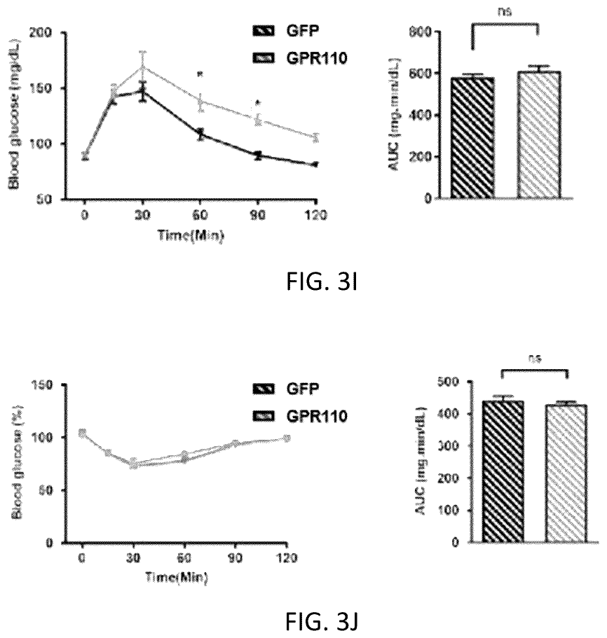

Based on the dramatic difference in expression levels of hepatic GPR110 before and after HFD treatment as shown in Example 1, downregulation of GPR110 in HFD-fed mice may be involved in the pathogenesis of fatty liver. To evaluate the impacts of high hepatic GPR110 level on liver metabolism, GPR110 was overexpressed in the hepatocytes of HFD-fed mice by liver-directed rAAV/thyroxine binding globulin (TBG)-mediated gene expression system (FIGS. 2A and 3A). In general, 8-week-old C57BL/6J male mice were housed in pathogen-free conditions at controlled temperature with a 12-hour light-dark cycle and access to food and water ad libitum. The 8-week-old male mice were divided into two groups and fed with high-fat diet (HFD, 20% protein, 45% fat, 35% carbohydrates, Research Diets Inc., New Brunswick, NJ, USA), or standard chow diet (STC, 18.3% protein, 10.2% fat, 71.5% carbohydrates, Research Diet Inc., New Brunswick, NJ, USA) in some other examples, for 8 weeks. The recombinant adeno-associated virus vector rAAV2/8 transduction was conducted as described previously (Lee et al, 2016; Cheng et al, 2022). Briefly, mice were tail vein injected with 3×10¹¹ rAAV2/8 vector harboring either green fluorescent protein (GFP) or GPR110. In other examples, for antisense oligonucleotide (ASO) delivery, validated ASOs against mouse GPR110 were provided by Ionis Pharmaceuticals and injected subcutaneously once a week at 5 mg/kg body weight to the mice. In some other examples, for SCD1 inhibitor delivery, MK-8245 (MedChemExpress, NJ, USA) was gavage at 10 mg/kg once a week. All measurements were carried out in a randomized order. Typically, glucose profile measurement was performed in terms of the blood glucose and insulin level by collecting the blood samples from the tip of the tail of the mouse models using a glucometer. For the glucose tolerance test (GTT), insulin tolerance test (ITT) and pyruvate tolerance test (PTT), mice were fasted overnight prior to intraperitoneal injection of glucose (1 g/kg body weight (BW)) (Sigma, St. Louis, MO), 0.75 U/kg BW insulin (Novolin R, Novo Nordisk, Bagsvaerd, Denmark) or 1 g/kg BW pyruvate (Sigma, St. Louis, MO). Blood glucose levels were measured from the tip of tail vein at 15, 30, 60, 90 and 120 minutes after injection.

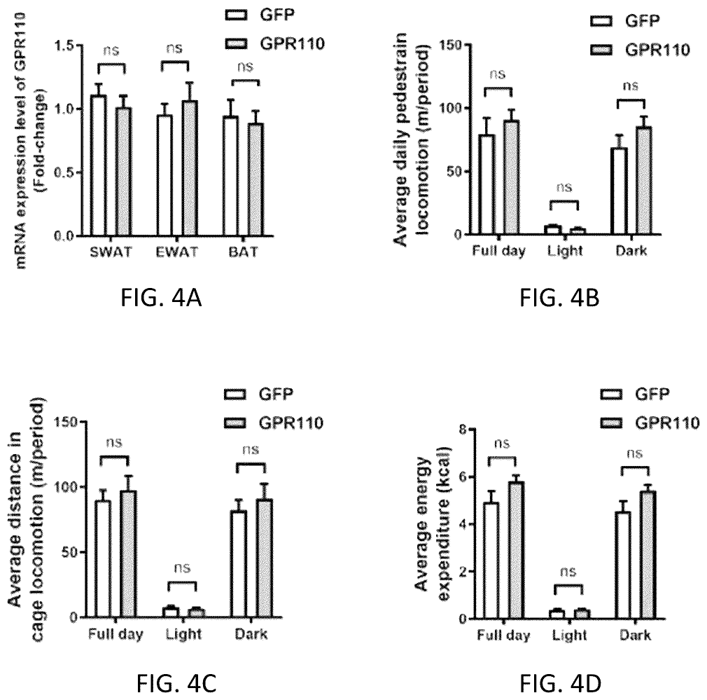

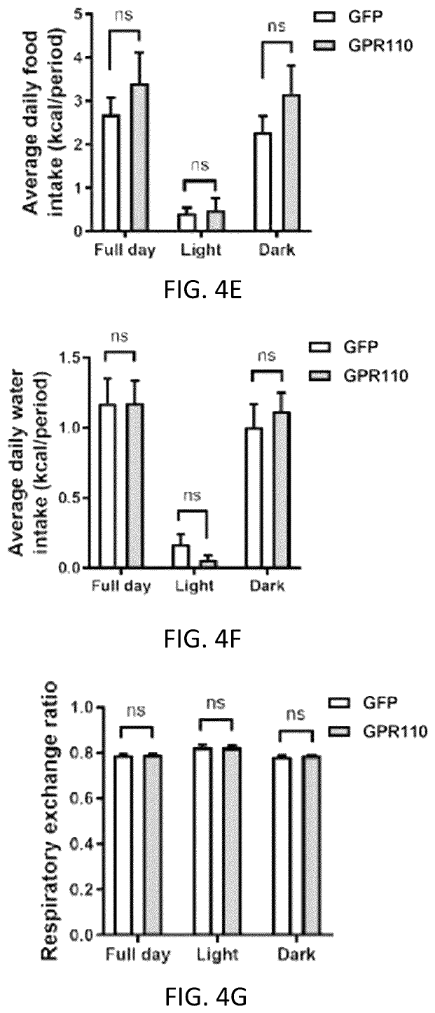

The overexpression of GPR110 in the livers of the mice were validated by RT-qPCR (FIGS. 2B and 3B) and Western blot analysis (FIG. 3C). The results confirm that rAAV-mediated GPR110 overexpression was solely in hepatocytes, but not in NPC, by Western blot analysis after cell fractionation (FIG. 2C). Renal and adipose GPR110 expression levels, on the other hand, were not affected by liver-directed rAAV/TBG-mediated gene expression (FIGS. 3C and 4A).

FIGS. 3D-3G show that overexpressing GPR110 in the liver of STC-fed mice did not affect body weight, fasting glucose level, fasting insulin level and homeostatic model assessment for insulin resistance (HOMA-IR). FIGS. 3H and 3I show that there was only a slight increase in glucose excursion curve in response to the GTT and hepatic glucose production induced by sodium pyruvate in PTT at several time points. On the other hand, no change in insulin sensitivity was observed by ITT between STC-fed rAAV-GFP and rAAV-GPR110 mice (FIG. 3J).

In contrast, under HFD treatment, rAAV-GPR110 mice gained more body weight (FIG. 2D), and body fat mass (FIGS. 2E-2F) than their rAAV-GFP controls. The HFD-fed rAAV-GPR110 mice also had higher fasting glucose level (FIG. 2G), fasting insulin level (FIG. 2H) and HOMA-IR (FIG. 2I). Worsen glucose tolerance was observed in HFD-fed rAAV-GPR110 mice (FIG. 2J). Overexpression of GPR110 in livers significantly increased hepatic glucose production induced in PTT (FIG. 2K). ITT showed that the glucose levels in HFD-fed rAAV-GPR110 mice remained insensitive at 30 to 60 minutes after injection of insulin as compared to their control HFD-fed rAAV-GFP littermates (FIG. 2L).

In addition, the rAAV-GPR110 mice were placed into metabolic cages to explore their locomotor activities (FIGS. 4B-4C), energy expenditure (FIG. 4D), food intake (Figure S3E), water intake (FIG. 4F) and respiratory exchange ratio (FIG. 4G). In brief, there were no difference in these metabolic parameters between the HFD-fed rAAV-GPR110 and rAAV-GFP littermates (FIGS. 4B-4G). Overall, a mild impairment in glucose homeostasis associated with overexpressing GPR110 in the livers of STC-fed mice was observed. More dramatical impairment was observed when the rAAV-GPR110 mice was challenged with HFD as compared to their rAAV-GFP controls.

### Example 3—Suppressing GPR110 Improves Glucose Homeostasis in HFD-Fed rAAV-GPR110 Mice

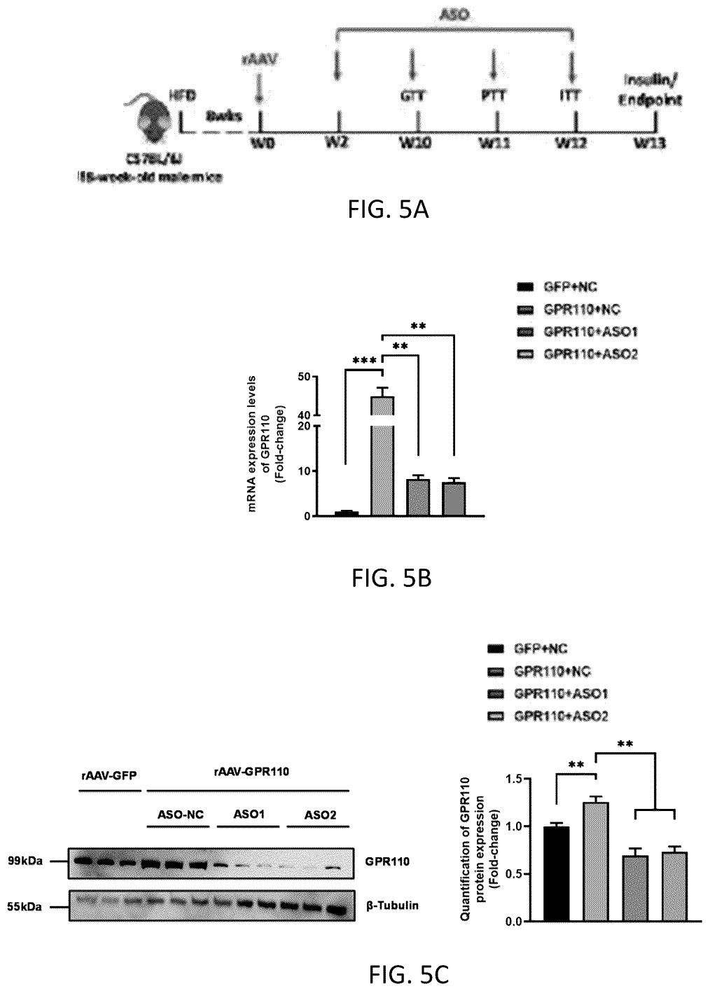

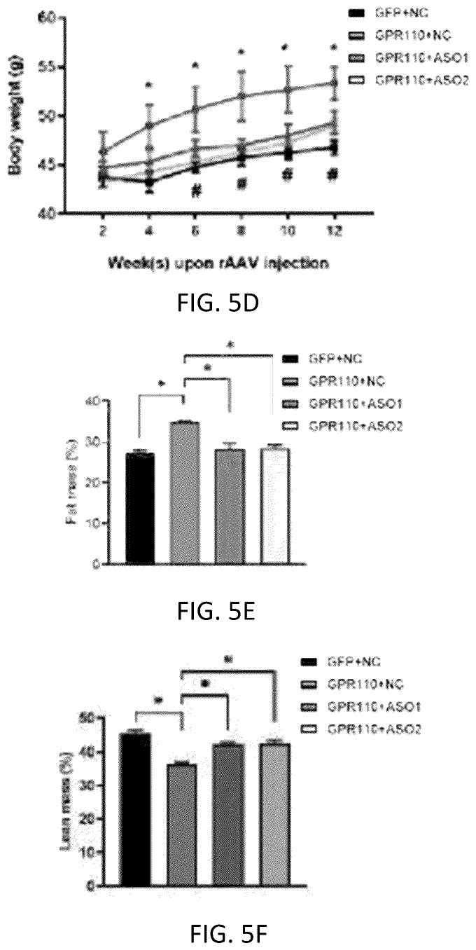

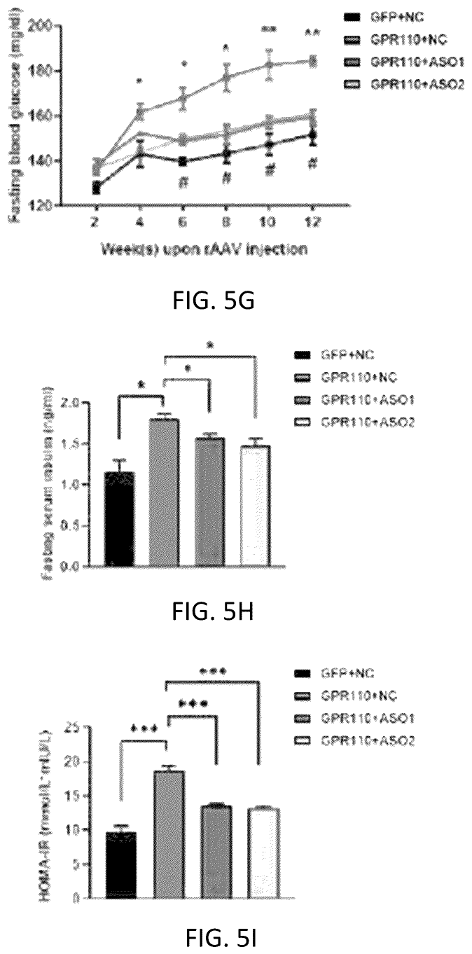

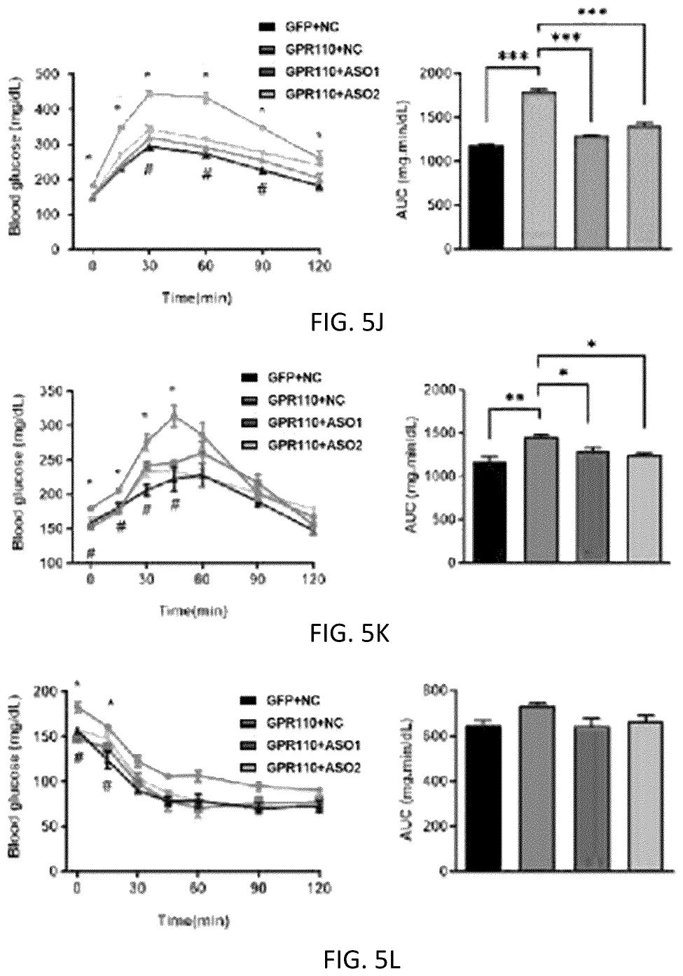

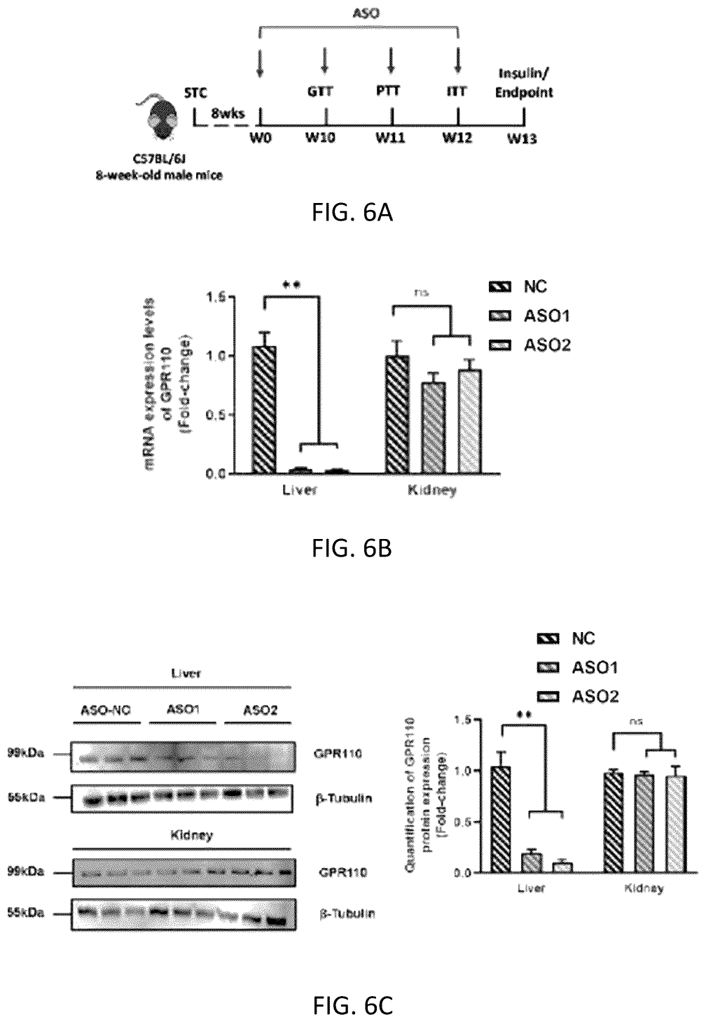

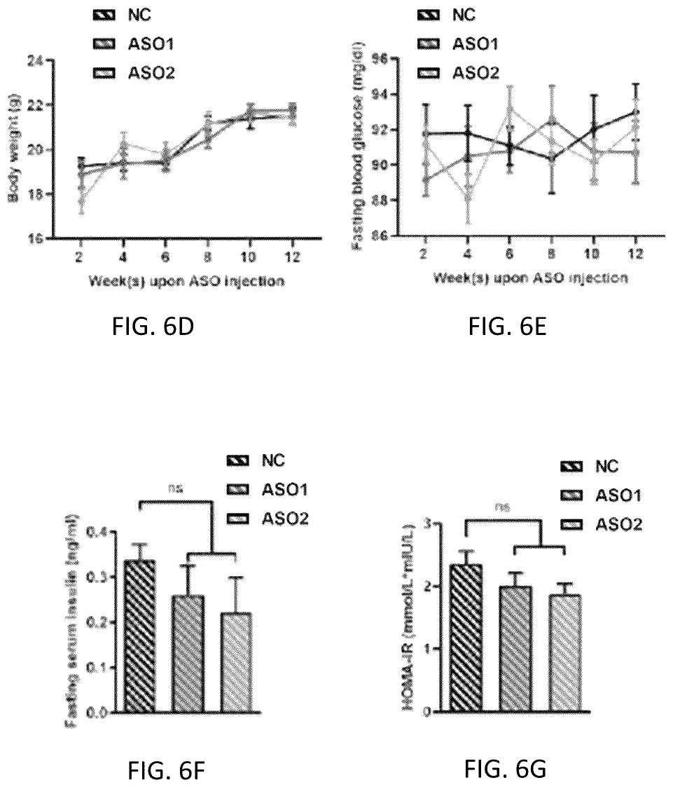

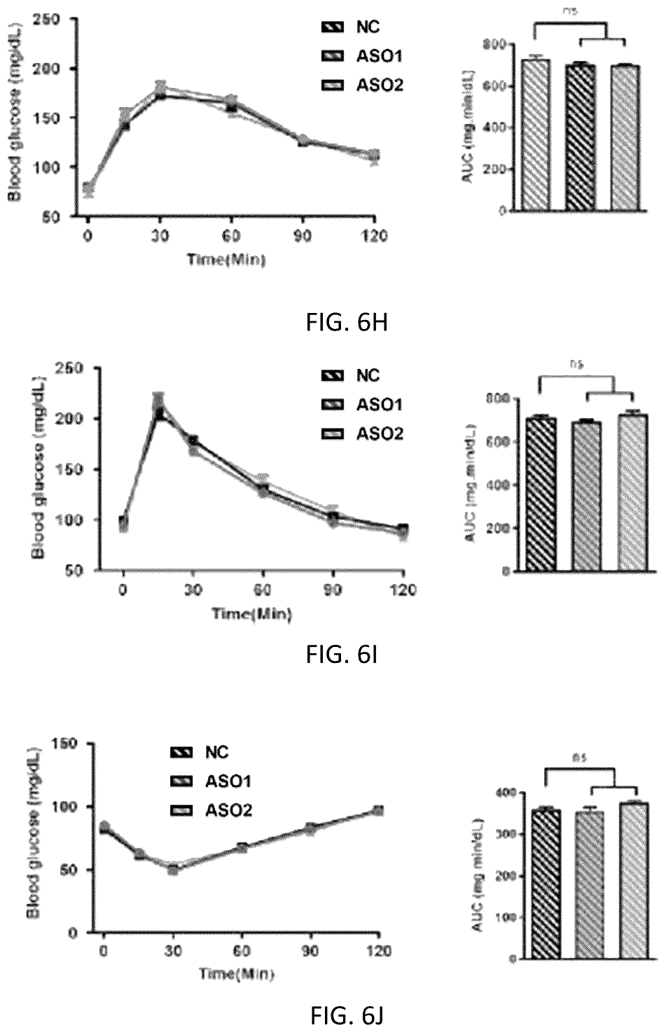

To confirm that the observations in Example 2 were due to the rAAV-mediated overexpression of hepatic GPR110 in HFD-fed mice, two N-acetylgalactaosamine (GalNAc) conjugated antisense oligonucleotides (ASO-GPR110s) that bind to different regions of GPR110 mRNAs were used in this example to knockdown the hepatic GPR110 expression in mice (FIGS. 5A and 6A). To avoid a false observation due to off-target effects, two different sequences of ASO were used. Chronic treatment of either ASO-GPR110s only lowered the hepatic, but not renal, GPR110 mRNA (FIGS. 5B and 6B) and protein (FIG. 6C) levels. It is due to the fact that liver hepatocytes abundantly and specifically express the asialoglycoprotein receptor that binds and uptakes circulating glycosylated oligonucleotides via receptor-mediated endocytosis. FIGS. 6D-6E show that knockdown hepatic GPR110 by ASO-GPR110s in STC-fed mice did not affect body weight and fasting glucose, but it slightly lowered insulin level (FIG. 6F) and HOMR-IR (FIG. 6G), as compared to their littermates injected with the negative control, scrambled antisense oligonucleotides (ASO-NC). No difference in the changes of glucose levels in GTT (FIG. 6H), PTT (FIG. 6I) and ITT (FIG. 6J) for both ASO-GPR110 and ASO-NC groups under STC feeding conditions.

In contrast, chronic ASO-GPR110 treatment for 4 weeks significantly decreased their body weight (FIG. 5D), fat mass ratio (FIGS. 5E-3F) fasting glucose level (FIG. 5G), fasting insulin level (FIG. 5H) and HOMA-IR (FIG. 5F) in HFD-fed rAAV-GPR110 mice. In addition, treatment of ASO-GPR110 improved glucose tolerance, pyruvate tolerance and insulin sensitivity in HFD-fed rAAV-GPR110 mice as demonstrated by GTT (FIG. 3J), PTT (FIG. 3K) and ITT (FIG. 3L) as compared to ASO-NC controls. In consistent to overexpressing GPR110 in livers, the depletion of hepatic GPR110 by ASOs improves glucose homeostasis in HFD-fed mice.

### Example 4—Treatment of ASO-GPR110s Alleviates Lipid Abundance and Liver Damage in HFD-Fed rAAV-GPR110 Mice

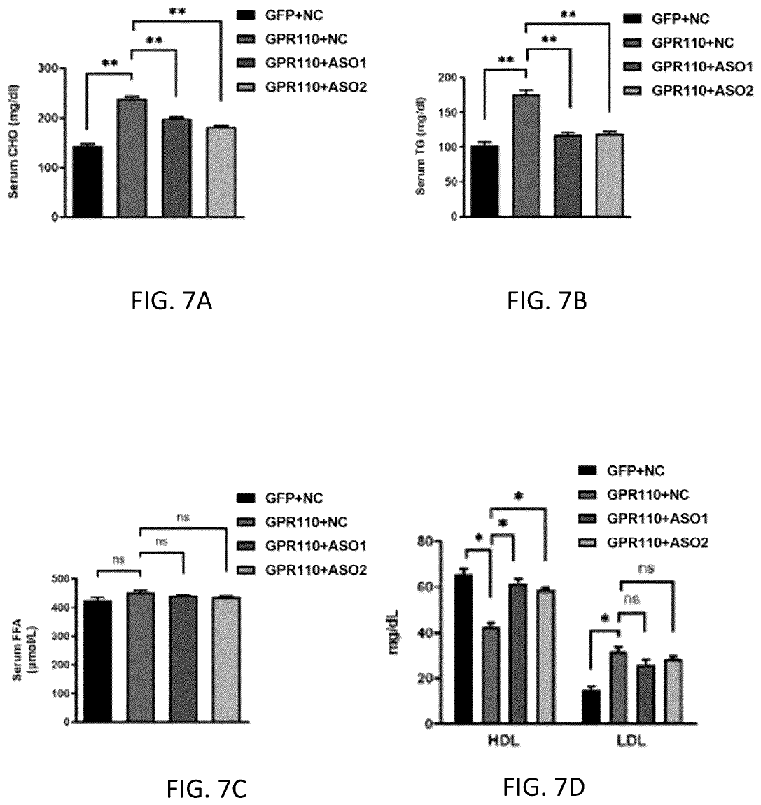

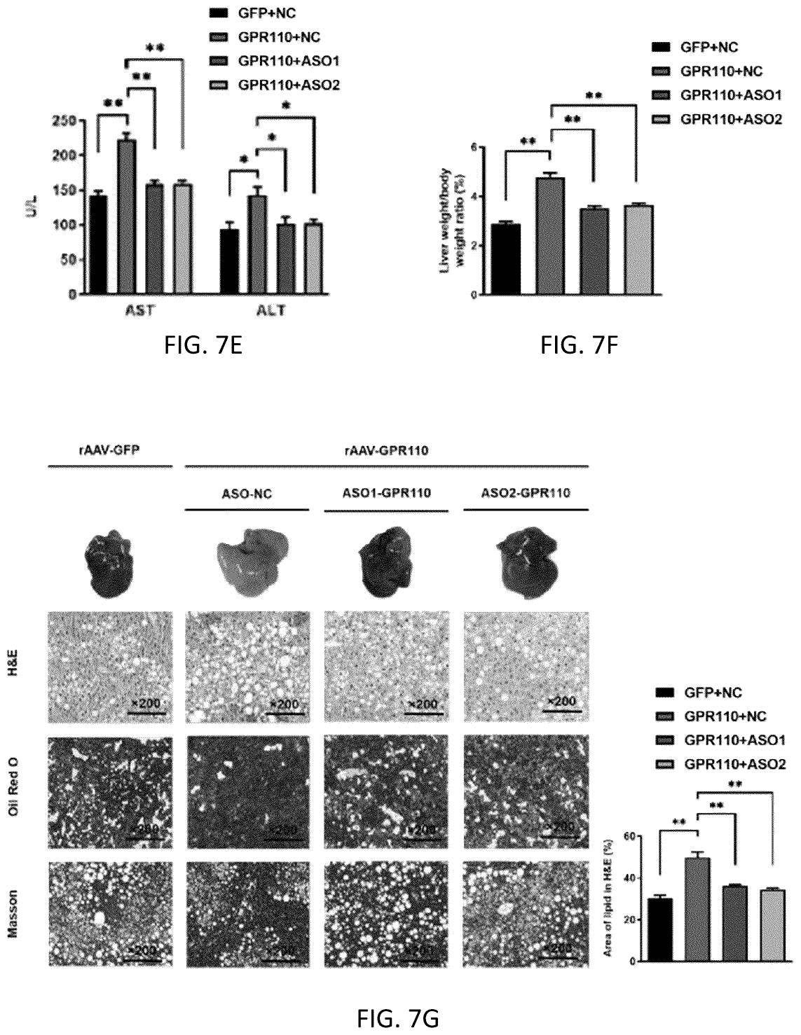

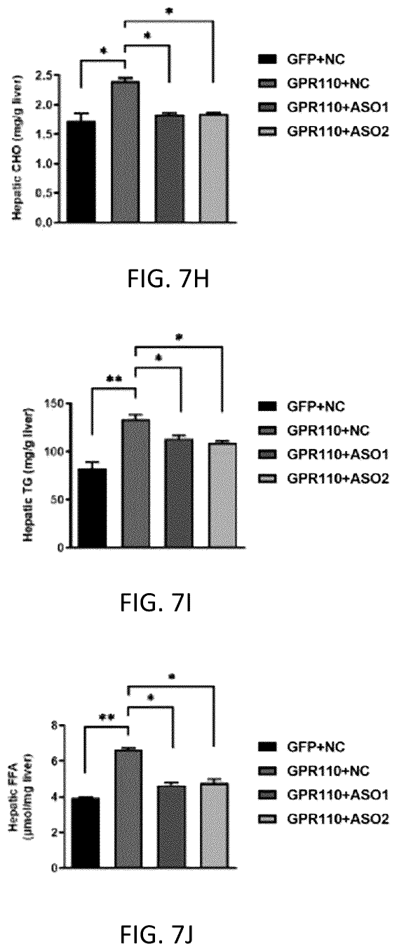

FIGS. 7A-7D show circulating lipid profiles of the mice. Typically, for plasma and hepatic lipid level, serum levels of triglyceride (TG) and total cholesterol (CHO) were measured using commercial kit (Biosino, biotechnology and science INC, China) according to the manufacturer's instructions. Hepatic lipids were extracted using Folch methodology and liver extract was dissolved in ethanol for TG and CHO measurements. Both serum and hepatic levels of free fatty acid (FFA) were measured using commercial kit (Solarbio, China).

FIGS. 7A-7C show that HFD-fed rAAV-GPR110 mice had higher circulating cholesterol (CHO) and triglyceride (TG) levels than HFD-fed rAAV-GFP littermates, but their circulating free fatty acid (FFA) levels were similar. High-density lipoprotein (HDL) cholesterol level was decreased, and low-density lipoprotein (LDL) cholesterol level was increased in HFD-fed rAAV-GRP110-NC mice as compared to chronic ASO-GPR110 treatment group (FIG. 7D).

FIG. 7E shows that chronic ASO-GPR110 treatment could lower circulating levels of the liver enzymes, aspartate transaminase (AST) and alanine aminotransaminase (ALT), which are markers of liver damage and hepatoxicity, in HFD-fed rAAV-GRP110-NC mice. Typically, for liver function assay, the alanine aminotransferase (ALT) and aspartate aminotransferase (AST) levels were measured in serum using commercial kits (Stanbio, USA).

After sacrificing the mice, their hepatic lipid profiles were also studies. The livers of HFD-fed rAAV-GRP110 mice were significantly heavier (FIG. 7F) and paler (FIG. 7G, upper panels) than the livers of their rAAV-GFP littermates and ASO-GPR110 treated rAAV-GRP110 mice. Consistent with these observations, HFD-induced lipid accumulation within hepatocytes were substantially more abundant in the livers of HFD-fed rAAV-GPR110 mice than the rAAV-GFP littermates as determined by haematoxylin and eosin (H&E) staining and Oil Red O staining (FIG. 7G, upper and middle rows of left panel). Moreover, based on the Masson's trichrome staining, more fibre extension and larger fibrous septa formation was observed for the liver samples of rAAV-GPR110 mice as compared to the livers from rAAV-GFP littermates (FIG. 7G, lower row of left panel). These alterations were remarkably reduced after ASO-GPR110 treatments (FIG. 7G) Like the circulating lipid profiles mentioned above, treatment of ASO-GPR110s for 8 weeks could improve the hepatic lipid profiles of rAAV-GRP110 mice in terms of CHO (FIG. 7H), TG (FIG. 7I) and FFA (FIG. 7J). Altogether, overexpression of hepatic GPR110 in mice is sufficient to perturb lipid metabolism and hence the progression of NAFLD, especially in obese subjects.

### Example 5—The Metabolic Dysregulation of rAAV-GRP110 Mice is Correlated to the Upregulation SCD1 Expression

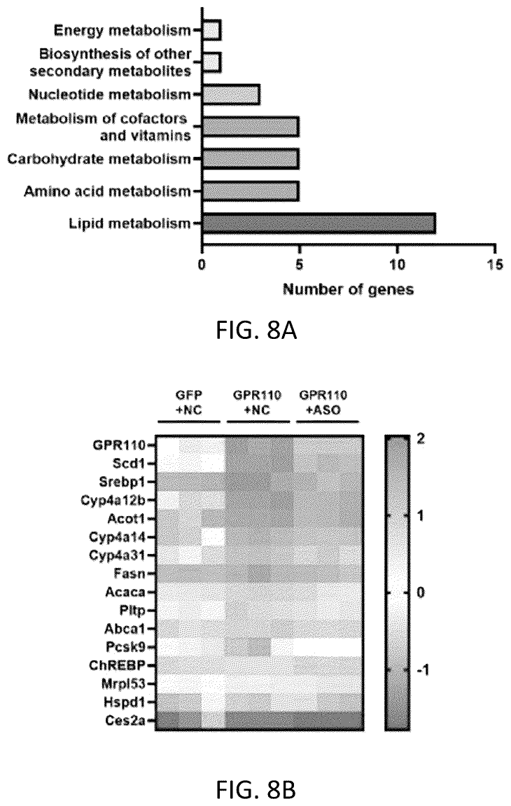

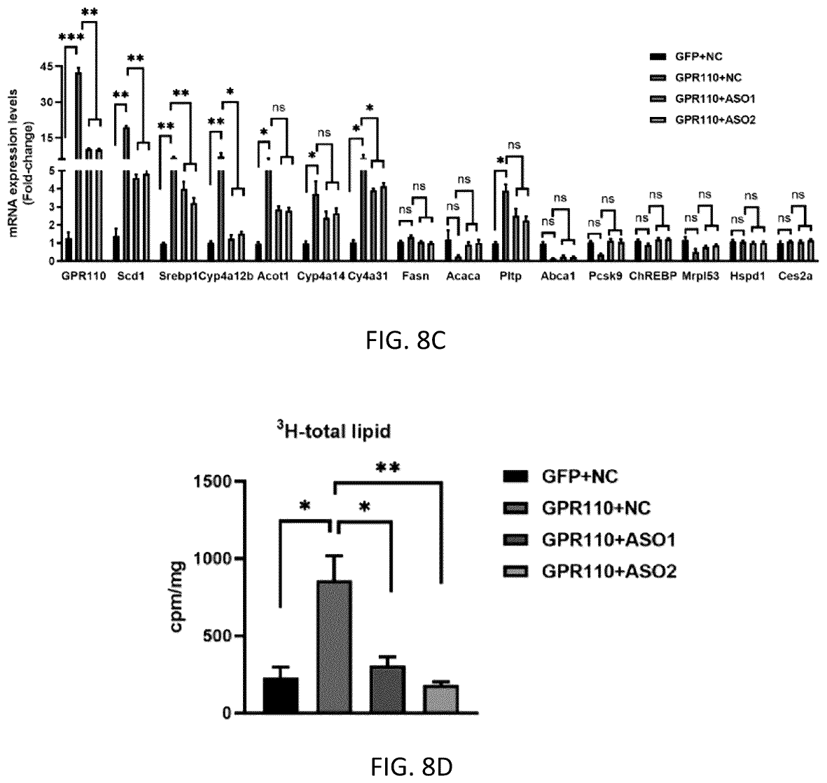

To reveal the molecular mechanism underlying the involvement of hepatic GPR110 in NAFLD development, RNA-sequencing analysis was performed on RNA samples extracted from the livers of HFD-fed ASO-NC treated rAAV-GPF, ASO-NC treated rAAV-GPR110 and ASO-GRP110 treated rAAV-GPP110 mice. In the search for the molecular processes for metabolisms, several lipid metabolism-related genes were altered (FIGS. 8A-8B). Typically, a heat map was created based on log₂ transformed counts from different samples. To be included in the heat map, genes were required to have at least 1000 counts, totaled over all samples, where and the standard deviation of the log₂ had to exceed two.

RT-qPCR was used to confirm the RNA sequencing results (FIG. 8C). Among them, stearoyl CoA desaturase 1 (SCD1) was of particular interest. SCD1 is a key lipogenic enzyme responsible for the rate-limiting step in the synthesis of monounsaturated fatty acids (MUFAs), such as oleate and palmitoleate, by forming double bonds in saturated fatty acids. MUFAs serve as substrates for the synthesis of various kinds of lipids and increases in SCD1 activity is involved in the development of NAFLD, hypertriglyceridemia, atherosclerosis, and diabetes. To confirm the role of GPR110 in lipogenesis, the de novo lipogenesis assay was performed. Typically, primary mouse hepatocytes were seeded into 6-well plates and washed the cells with warm PBS twice one night prior to the assay. The hepatocytes were changed to serum starvation medium with 100 nM insulin and incubated overnight at 37° C. The lipogenesis medium made up of 100 nM insulin, 10 μM cold acetate and 0.5 μCi ³H-acetate was added to the hepatocytes and incubated for 2 hours. Cells were then washed with PBS twice and 0.1 N HCl was used to lyse cells. Lipids were extracted by addition of 500 μl of 2:1 chloroform-methanol (v/v). Total lipid content was then calculated by measuring ³H activity.

Hepatocytes isolated from the rAAV-GPR110 group showed the highest lipogenesis activity, while those from the control rAAV-GFP group had lower activity (FIG. 8D). Furthermore, the lipogenic activity decreased in the hepatocytes isolated from rAAV-GPR110 mice treated with GPR110-specific ASOs compared to the control group (FIG. 8D). In agreement with the RT-qPCR data mentioned above, the results revealed a direct correlation between the level of GPR110 expression and lipogenic activity.

### Example 6—The Upregulation of SCD1 Expression in Liver is Driven by the Presence of GPR110

To confirm SCD1 expression is induced by GPR110, in vitro assays were performed by using adenovirus-mediated GPR110 expression system (ADV-GPR110) to overexpress GPR110 in primary hepatocytes isolated from STC-fed mice. Typically, primary hepatocytes from different groups of mice were isolated using a two-step perfusion method. For adenovirus viral infection, serum-starved cells were infected with adenoviruses carrying mouse GPR110 cDNA to overexpress GPR110. Similar adenoviral vectors encoding the green fluorescent protein (GFP) gene were used as controls.

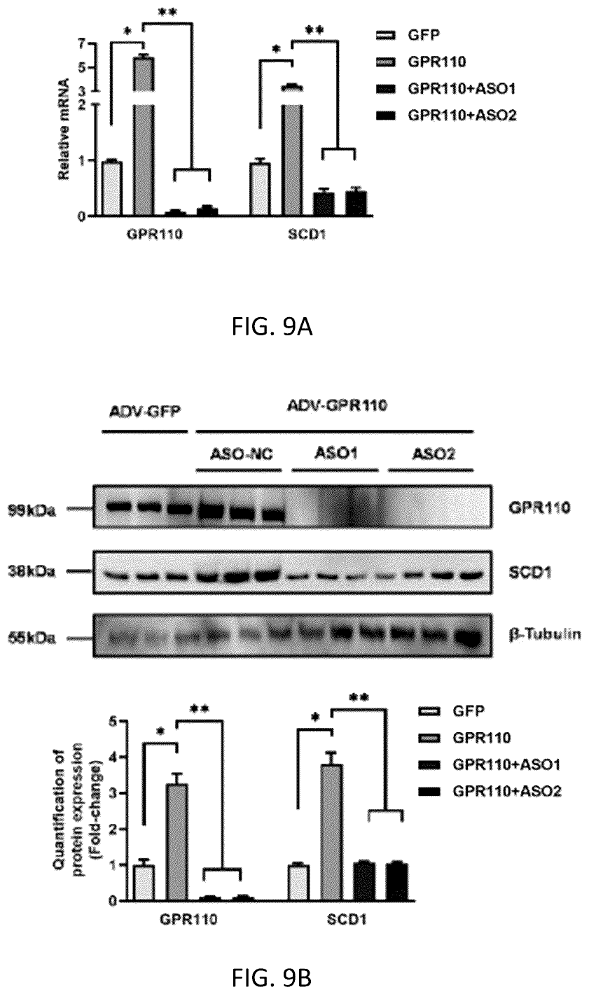

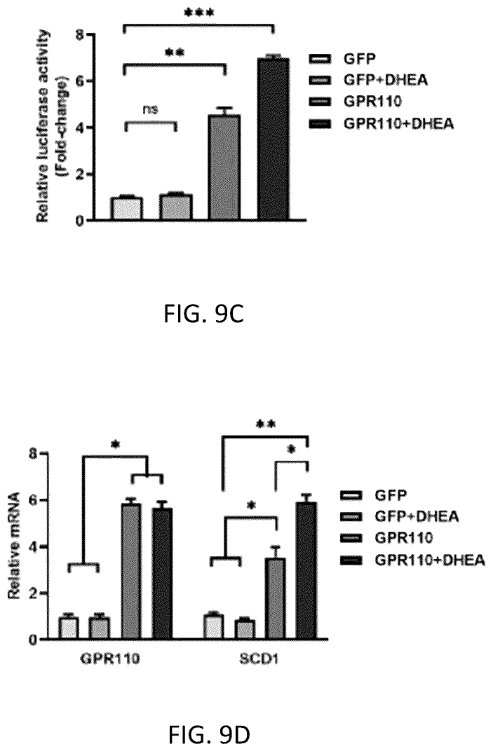

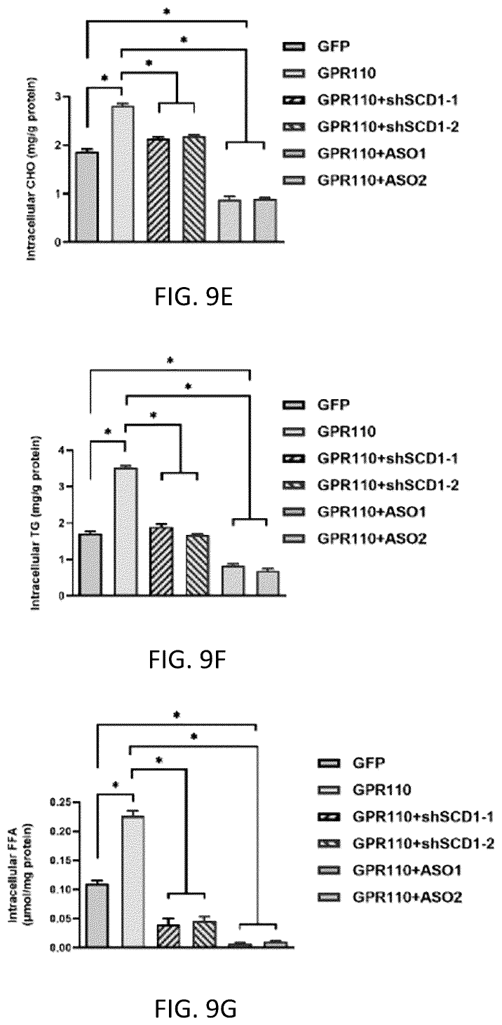

After infection, the expressions of SCD1 mRNAs (FIG. 9A) and protein (FIG. 9B) were dramatically induced, but not in control group which was treated with ADV-GFP. In addition, the GPR110 specific ASOs can not only knockdown the GPR110 expression, but also induced the SCD1 expression by increasing GPR110 in the ADV-GPR110 primary hepatocytes (FIGS. 9A-6B). In vitro luciferase reporter assay was performed to further validate the expression of SCD1 is transcriptional regulated by GPR110. Plasmid harbouring luciferase gene driven by the mouse SCD1 promoter (−2000 to +100) was constructed to transfect into HEK293 cells. Typically, HEK293 cells were cultured in DMEM supplemented with 10% FBS, 100 U/ml penicillin and 100 μg/ml streptomycin at 37° C. and 5% CO₂, and then seeded in 6-well plates followed by transfection with pGL3-SCD1 promoter and adenoviral vector expressing either GPR110 (ADV-GPR110) or GFP (ADV-GFP) by using the transfection reagent (#E4981, Promega, WI, USA). There was no change of luciferase activity of pGL3- SCD1 promoter-luciferase transfected HEK293 cells under the treatment of GPR110 ligand DHEA, unless the HEK293 cells were pre-infected with adenovirus overexpressing GPR110 (ADV-GPR110; FIG. 9C). The overexpression of GPR110 in ADV-GPR110 infected cells and inductions of SCD1 mRNA expression by treatment of DHEA were also validated by RT-qPCR (FIG. 9D). The changes in hepatocyte lipid profiles by the expression level of GPR110 and SCD1 were also checked. In agreement with the in vivo data, the overexpression of GPR110 increased the intracellular CHO (FIG. 9E), TG (FIG. 9F) and FFA (FIG. 9G). Their increases could be completely repressed by ASO against GPR110 (FIGS. 9E-9G) and partially repressed by overexpressing SCD1 specific shRNAs (FIGS. 9F-9G). In summary, the transcription level of SCD1 is regulated by GPR110. GPR110 enhances the lipid accumulation by inducing SCD1 expression.

### Example 7—Inhibition of SCD1 in rAAV-GPR110 Mice Partially Attenuates Most Metabolic Dysregulations Especially Lipid Profiles

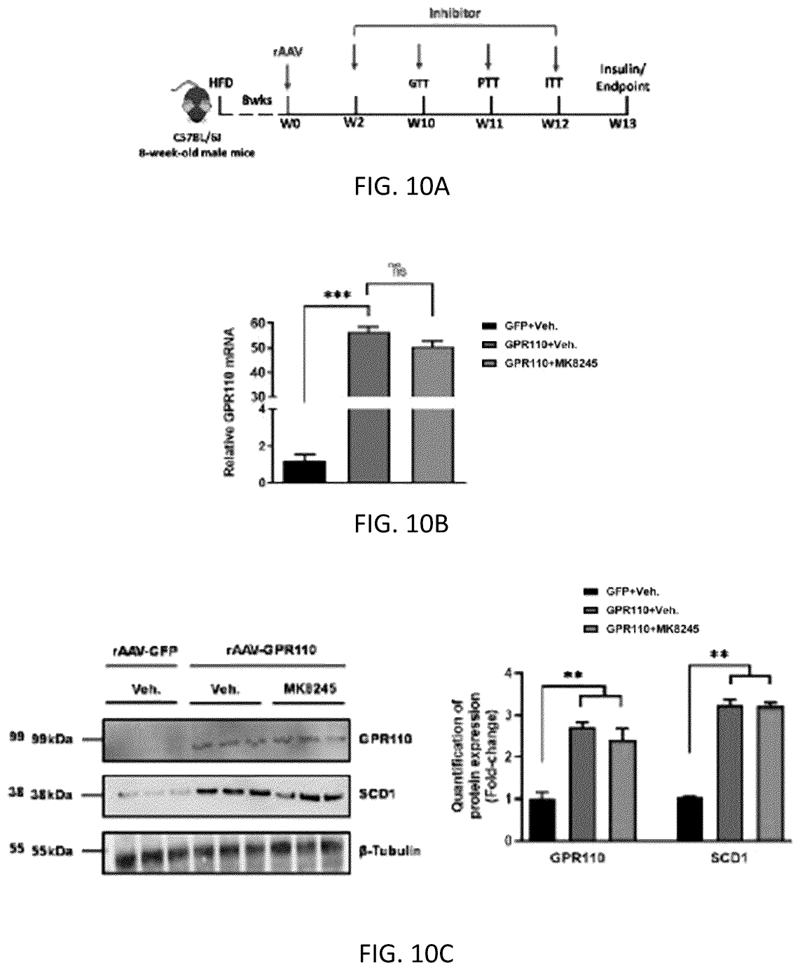

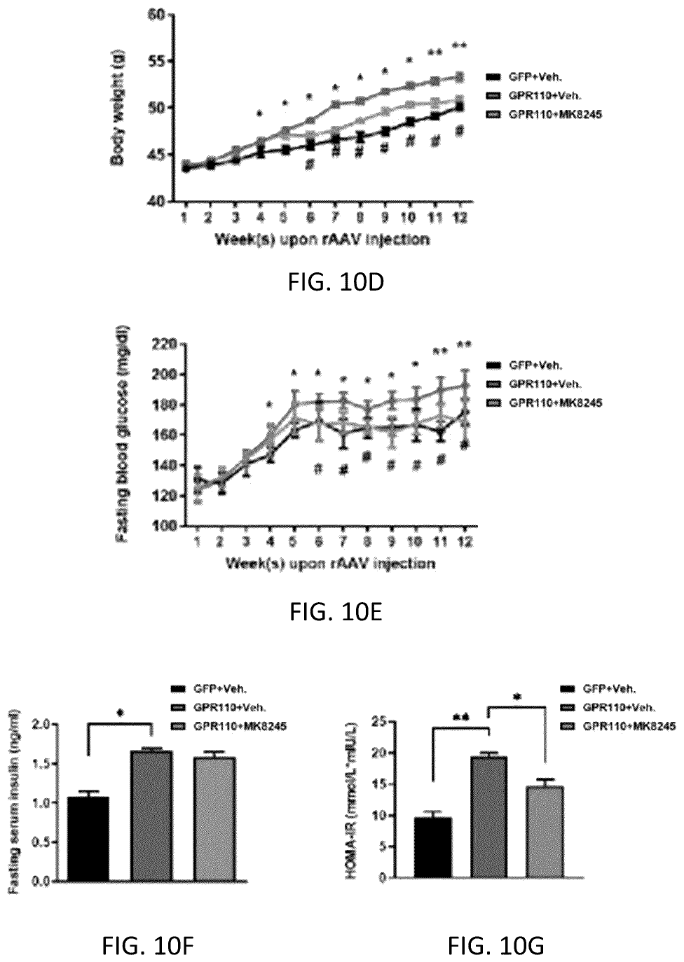

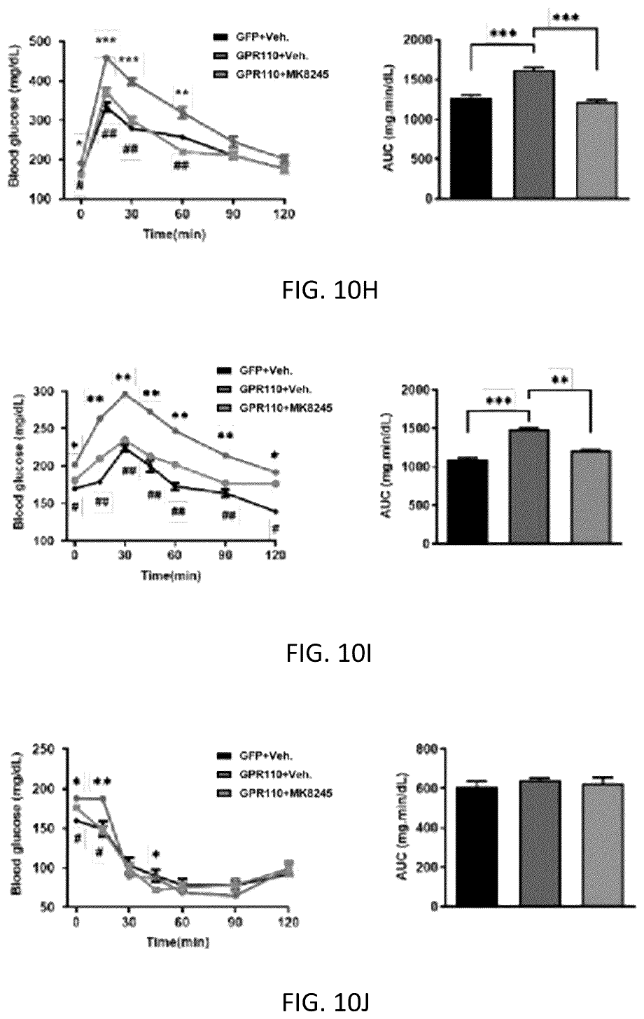

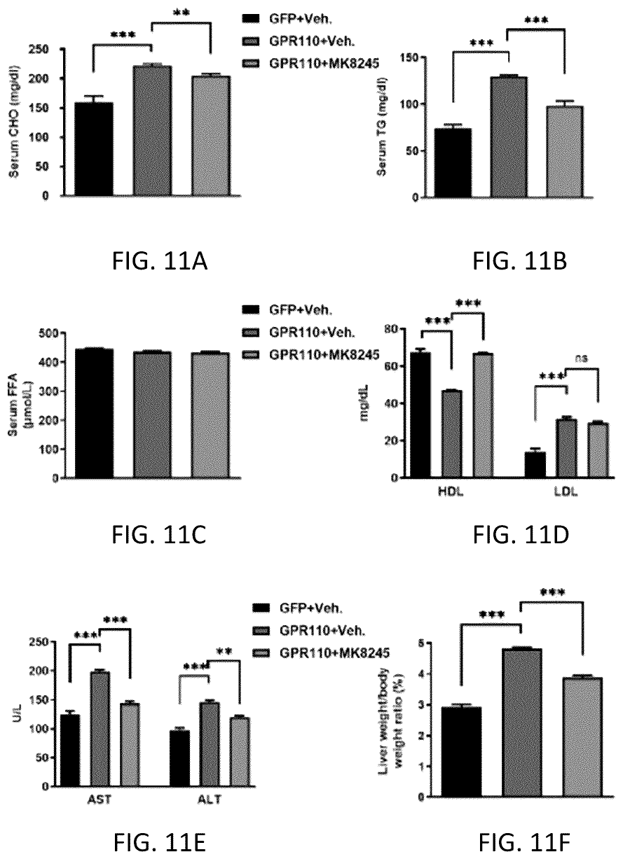

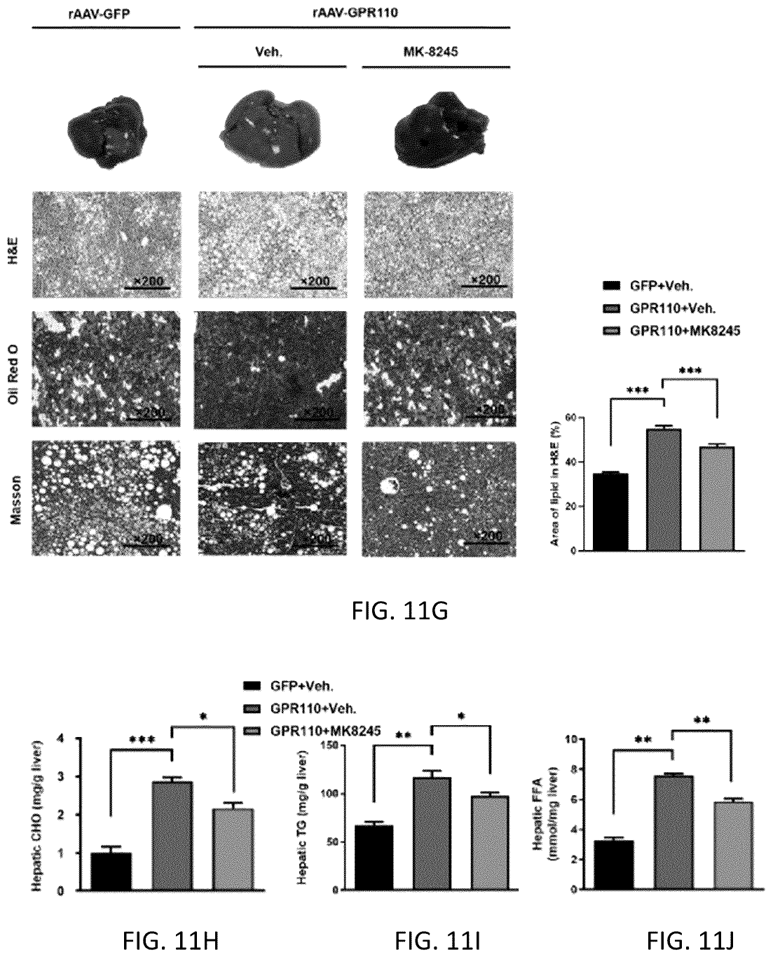

To examine whether the up-regulation of hepatic SCD1 leads to metabolic dysregulation in rAAV-GRP110 mice, a liver-specific SCD1 inhibitor, MK8245, was used to alleviate the metabolic dysregulation by overexpressing GPR110 in HFD-fed rAAV-GRP110 mice (FIG. 10A). Chronic treatment of MK8245 for 11 weeks did not affect the expression of GPR110 mRNA (FIG. 10B) and protein (FIG. 10C) levels in rAAV-GRP110 mice. In agreement with previous studies showing that the chronic treatment of this SCD1 inhibitor improves various metabolic parameters including lipid and glucose profiles in various animal models (Oballa et al., 2011), treatment of MK8245 lowered the body weight (FIG. 10D), improved glucose homeostasis in term of fasting glucose level (FIG. 10E) and HOMR-IR (FIG. 10G), and performance in GTT (FIG. 10H) and PTT (FIG. 10I) of HFD-fed rAAV-GRP110 mice as compared to untreated littermates. But there was no change in insulin sensitivity as demonstrated by ITT (FIG. 10J). MK8245 treatment also lowered the circulating CHO (FIG. 11A) and TG (FIG. 11B) levels almost to the levels of HFD-fed rAAV-GFP mice, but there was no change in circulating FFA level (FIG. 11C). A relatively higher HDL can be found in the MK8245 group but there were no differences detected regarding the LDL level (FIG. 11D). The AST and ALT levels were also alleviated in the MK8245 group compared to the rAAV-GPR110 littermates (FIG. 11E). MK8245 treatment partially reduced the liver weight (FIG. 11F), degree of paleness, severity of fibrosis (FIG. 11G) and lipid accumulations (FIGS. 11H-11J). To conclude, treatment of MK8245 could improve the lipid profiles and alleviate metabolic dysregulation caused by overexpression of hepatic GPR110 in mice.

### Example 8—Expression of GPR110 in Liver is Closely Associated with Hepatic Steatosis in NAFLD Patients

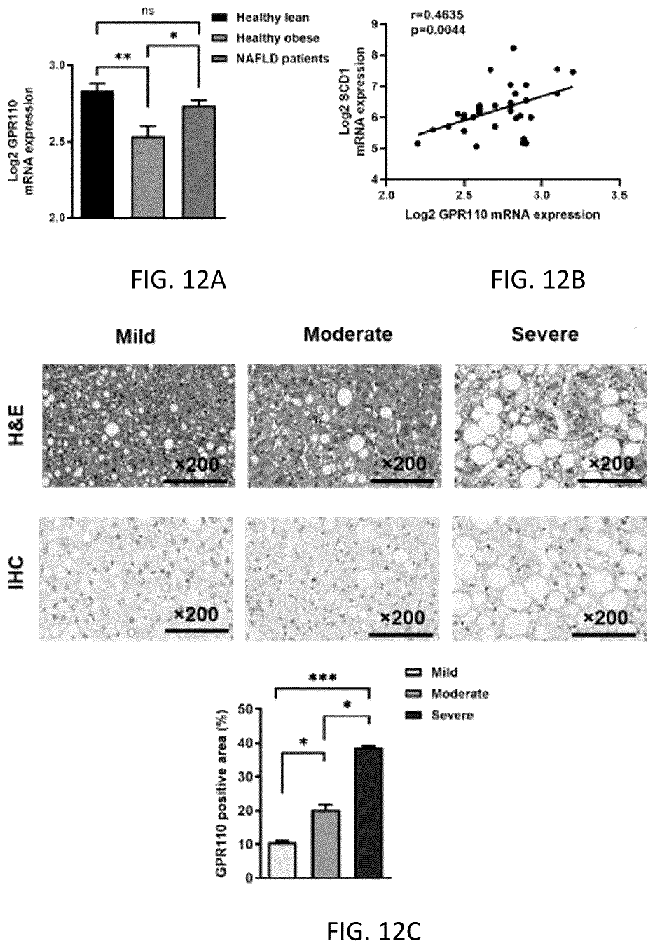

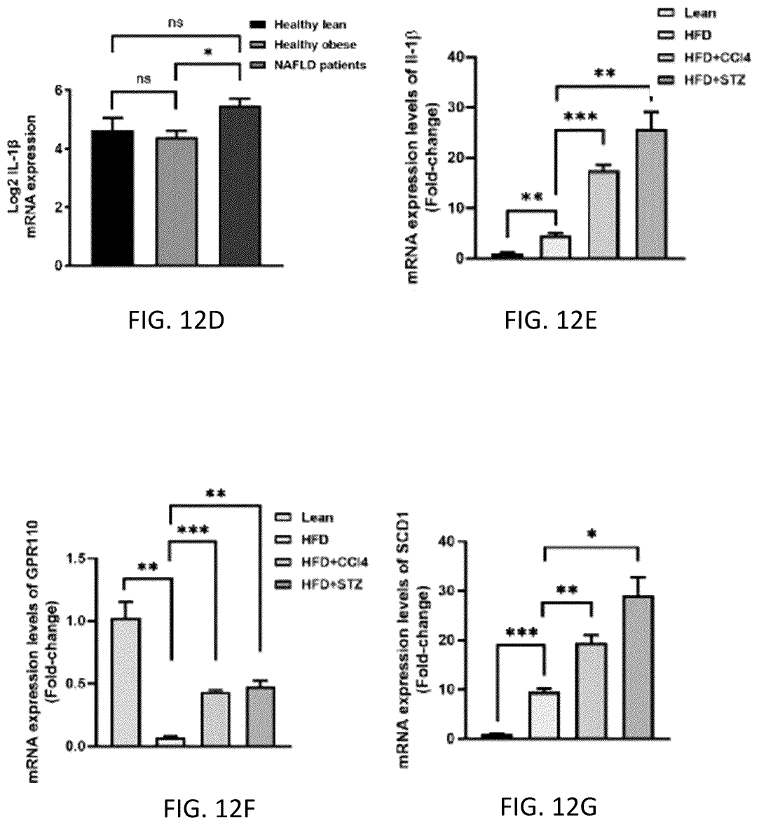

To evaluate the clinical relevance of our findings in mice, the expression level of GPR110 in human liver from a publicized transcriptome dataset Gene Expression Omnibus (GEO Profile # GDS4881) with human liver biopsy of different phases from control to NAFLD. Typically, liver biopsy specimens were collected from 9 biopsy-proven NAFLD patients. Healthy obese subjects without NAFLD had lower GPR110 mRNA expression than healthy lean subjects, but obese NAFLD subjects had similar GPR110 mRNA expression level as healthy lean subjects (FIG. 12A). Subsequently, by using the same transcriptome dataset, the correlation in the expression level of GPR110 and SCD1 was studied. The expression level of GPR110 was positively correlated with SCD1 in the liver (r=0.4635, P<0.05; FIG. 12B).

To verify the observation, immunohistochemistry staining was performed with liver sections from biopsy-proven patients with mild, moderate, and severe NAFLD, respectively (Table 4). Typically, liver sections with H&E staining (top row in upper panel of FIG. 12C) were subjected to histological evaluation of steatosis. Simple steatosis was defined by the presence of macrovascular steatosis affecting at least 5% of hepatocytes without inflammatory foci and evidence of hepatocellular injury in the form of hepatocyte ballooning. Individuals with a heavy alcohol-drinking history (≥40 g/day for up to 2 weeks), drug-induced liver disease and hepatitis virus infection were excluded from the study. Clinical parameters of individuals were summarized in Table 4.

TABLE 4

| NAFLD stage | Mild | Moderate | Severe |
|---|---|---|---|
| Age | 49.00 ± 15.10 | 47.33 ± 15.89 | 25.67 ± 5.66 |
| Gender (Female/Male) | 2/1 | 0/3 | 2/1 |
| Body Weight (kg) | 66.67 ± 14.41 | 65.90 ± 15.79 | 79.00 ± 1.41 |
| Body Height (m) | 1.60 ± 0.13 | 1.70 ± 0.10 | 1.68 ± 0.01 |
| BMI (kg/m²) | 25.65 ± 2.06 | 22.59 ± 3.84 | 28.08 ± 0.27 |
| Fasting Glucose (mmol/L) | 5.07 ± 0.35 | 6.42 ± 1.42 | 5.51 ± 1.48 |
| SBP (mmHg) | 122.33 ± 11.24 | 114.33 ± 26.50 | 130.67 ± 0.71 |
| DPB (mmHg) | 76.33 ± 17.04 | 75.33 ± 17.01 | 94.00 ± 25.46 |
| Total Cholesterol (mmol/L) | 4.29 ± 0.90 | 3.32 ± 0.52 | 3.93 ± 1.18 |
| Triglycerides (mmol/L) | 1.29 ± 0.47 | 1.99 ± 1.44 | 1.06 ± 0.26 |
| HDL (mmol/L) | 1.38 ± 0.23 a | 0.82 ± 0.25 | 1.05 ± 0.14 |
| LDL (mmol/L) | 2.39 ± 0.68 | 1.82 ± 0.21 | 2.51 ± 1.28 |
| ALT (U/L) | 38.33 ± 27.39 | 87.33 ± 41.53 | 91.33 ± 52.33 |
| AST (U/L) | 16.33 ± 6.81 a, b | 46.00 ± 16.00 | 51.33 ± 16.26 |
| AST/ALT | 0.63 ± 0.40 | 0.63 ± 0.40 | 0.60 ± 0.21 |
| γGGT (U/L) | 117.67 ± 163.10 | 47.67 ± 6.43 | 100.67 ± 32.53 |

Abbreviations: BMI, body mass index; HOMA-IR, homeostasis model assessment of insulin resistance; SBP, systolic blood pressure; DBP, diastolic blood pressure; HDL, high density lipoproteins; LDL, low density lipoproteins; ALT, alanine aminotransferase; AST, aspartate transaminase; γGGT, γ-glutamyl transpeptidase. Data represent as mean ± SEM;
a, significant difference between mild and moderate;
b, significant difference between mild and severe, no significant differences among the rest groups

The degree of steatosis was determined by non-alcoholic steatohepatitis clinical research network (NASH CRN) scoring system. Immunostaining analysis demonstrated that hepatic expression of GPR110 protein was higher in the ones with severe steatosis than those with lower degree of NAFLD (FIG. 12C). These data collectively suggest that GPR110 expression level correlates to hepatic steatosis in humans as well.

To investigate the potential reason for higher hepatic GPR110 expression levels in NAFLD patients than in healthy obese individuals, a multiple-hit hypothesis was formulated, which suggests that liver inflammation may alter gene expression during NAFLD pathogenesis. Supporting this hypothesis, it is found that the mRNA level of IL-1β, a key mediator of low-grade inflammation during NAFLD was significantly higher in NAFLD patients than in healthy obese individuals in the GEO (Profile # GDS4881) (FIG. 12D). To validate this hypothesis, HFD-fed mice were treated with either CCl4 or STZ to accelerate and exacerbate their NAFLD pathogenesis , and measured the mRNA level of the hepatic inflammation marker, IL-1β. After treatment with either CCl4 or STZ, the expression of IL-1 mRNA in their livers increased 4 to 5-fold compared to the respective value of untreated HFD-fed mice (FIG. 12E). Notably, the expression of hepatic GPR110 mRNA was also significantly increased after CCl4 or STZ treatment compared to the untreated HFD-fed mice (FIG. 12F). Additionally, the SCD1 mRNA expression level in the CCl4 or STZ-treated HFD-fed mice was significantly higher than the respective value of untreated HFD-fed mice (FIG. 12G). These results suggest that inflammation induced by either CCl4 or STZ treatment can increase the expression of hepatic GPR110, in addition to SCD1, which is known to be involved in hepatic lipid metabolism.

### Example 9—Small Interference RNA (siRNA) Significantly Reduces GPR110 in Human Liver Cells

To validate the feasibility of using RNA interference (RNAi) techniques to reduce human hepatic GPR110 expression, four different human cell lines, HepG2, Hep3B, Huh7, and L-O2, were transfected with four different siRNA each having a nucleic acid sequence for targeting different positions on GPR110 mRNA (or ADGRF1 mRNA represented by SEQ ID NO: 39) as set forth in Table 5.

TABLE 5

| | Target | | SEQ ID |
|---|---|---|---|
| Name | position | Sequence | NO |
| siGPR110-1 | 61-83 | AAGAACTCATTGTGAATAAGAAA | 35 |
| siGPR110-2 | 280-302 | TTCTGCTATATACTCCAAATATG | 36 |
| siGPR110-3 | 695-717 | GAGATATCTTTGCACAGATAACA | 37 |
| siGPR110-4 | 1318-1340 | AACAGCATAAGAAGTGATTGAGC | 38 |

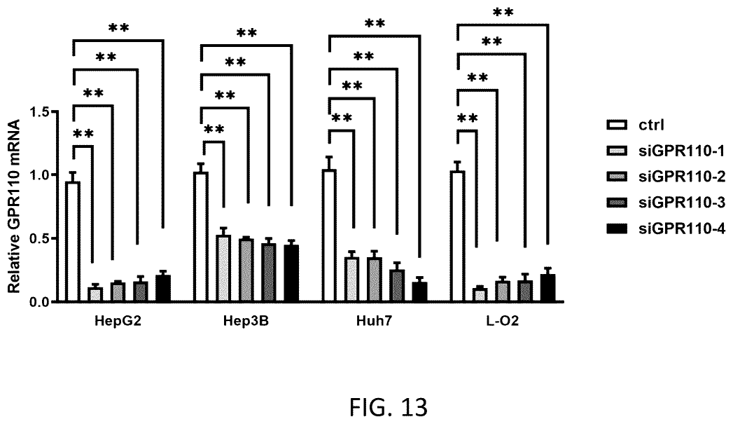

The siRNAs were transfected into the cells using Lipofectamine reagent according to the corresponding manufacturer's protocol. After a 48-hour transfection period, cells were collected, and reverse transcription quantitative polymerase chain reaction (RT-qPCR) was performed to measure the GPR110 mRNA levels. The results are shown in FIG. 13, in which all four siRNAs significantly reduced the expression levels of GPR110 in the different human liver cell lines, as compared to control.

As seen from FIG. 13, RNAi methods such as siRNA can effectively suppress GPR110 mRNA expression in human liver cells, providing an insight to develop into targeted therapeutics to NAFLD based on gene silencing mechanism. Besides ASO described in Examples 3-6 and siRNA in this example, it should be understood that other feasible or practical gene silencing mechanism such as shRNA, RICS, CRISPR with Cas9, etc. that can cleave or edit target mRNA, destabilize or modulate mRNA transcription or even its translation into proteins can also be used.

Taking the four siRNAs depicted in Table 5 as an example, the design of the RNA sequence for gene silencing of human GPR110 mRNA is based on a number of selection criteria such as accessibility of a target region, conservation of the target sequence, selectivity and specificity of the target sequence, relative position to protein translation start site, any disruption of protein function or mRNA splicing processes, sufficient GC content and desirable secondary structure in favor of siRNA binding and stability, and repeatability in application of multiple siRNAs, etc. More specifically, the target position on the mRNA of interest should be readily accessible to the concerned siRNA in terms of its binding and subsequent RNAi activity. In other words, regions with undesirable secondary structures, strong protein binding, or other hinderance that will limit the effectiveness of the siRNA should be avoided. Extreme GC-rich or GC-poor regions and strong secondary structure will likely interfere siRNA hybridization and its RNAi activities. In addition, the sequence of the target region on the mRNA of interest is preferably a conserved region across different variants or isoforms of the interested mRNA, so that the efficiency and specificity of the interested siRNA to knockdown the mRNA of interest across various cell types of the same phenotype or organisms of the same or similar genera can be increased. The target region should also have minimal sequence similarity to other non-target regions, genes or transcripts to avoid off-target effects, so as to minimize undesirable effects on the transfected cells or subject due to non-specific gene silencing by the siRNA. The target position is often selected in the 3′ untranslated region (UTR) of the interested mRNA. However, sometimes 5′ UTR can also be selected as the target position, depending on any specific characteristics of the target gene to be silenced and its regulatory elements. Other considerations include whether the target region may involve in protein translation of functional domains and/or exon-exon junctions required for mRNA splicing, and also whether the target region can be identified when multiple siRNAs are used at the same time.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

The present invention provides a clear gene target and its downstream mechanism for regulating lipid metabolism in liver specifically arising from NAFLD. Since the pathophysiology of NAFLD is still not well understood, but it is widely accepted that multiple factors, including inflammation, contribute to its acceleration and aggravation, to explain the observation that hepatic GPR110 expression levels in NAFLD patients are higher than in healthy obese individuals, key inflammation markers in the corresponding mouse models have been studied, and IL-1β and GPR110 expression levels are shown to be dramatically increased in subjects with more severe NAFLD conditions. It is interesting to note that the mechanism by which hepatic GPR110 transcription is repressed in healthy obese individuals whilst induced in NAFLD patients remains to be explored. In addition, although is commonly associated with NAFLD, a relatively high proportion of lean Asians can also develop NAFLD. Whether the expression levels of hepatic GPR110 mRNA in these “lean NAFLD” patients are higher than in lean healthy controls is worthwhile to explore. This could provide further insights to the role of hepatic GPR110 in NAFLD pathogenesis and help identify potential therapeutic targets specific for the treatment of NAFLD in lean individuals.

What is claimed is:

1. A method for treating non-alcoholic fatty liver disease (NAFLD), conditions and symptoms associated therewith comprising administering to a subject a composition comprising therapeutic nucleic acids to reduce G protein-coupled receptor 110 (GPR110) expression in liver of the subject.

2. The method of claim 1, wherein the composition is administered via intravenous injection, subcutaneous injection, or oral administration.

3. The method of claim 1, wherein the therapeutic nucleic acids are capable of gene silencing of GPR110.

4. The method of claim 3, wherein the therapeutic nucleic acids comprise small interfering RNA (siRNA), short-hairpin RNA (shRNA), micro RNA (miRNA), RNA induced silencing complex (RICS), or a complex of clustered regularly interspaced short palindromic repeats (CRISPR) with CRISPR-associated protein 9 (Cas9).

5. The method of claim 1, wherein the therapeutic nucleic acids are hepatic GPR110-specific antisense oligonucleotides (ASOs).

6. The method of claim 1, wherein the therapeutic nucleic acids are siRNAs each having a nucleotide sequence selected from one of SEQ ID NOs: 35-38.

7. The method of claim 1, wherein the conditions or symptoms associated with the NAFLD comprise hepatic steatosis, and chronic noncommunicable diseases (NCDs), non-alcoholic steatohepatitis (NASH) with fibrosis, cirrhosis and hepatocellular carcinoma (HCC), wherein the NCDs comprise type 2 diabetes mellitus (T2DM), cardiovascular disease (CVD), hypertriglyceridemia, atherosclerosis, and chronic kidney disease (CKD).

8. The method of claim 1, wherein the subject comprises non-human animals and humans.

9. The method of claim 1, further comprising a viral vector.

10. The method of claim 9, wherein the viral vector comprises adenoviral, adeno-associated viral, retroviral, or lentiviral vector.

11. A pharmaceutical composition comprising therapeutic nucleic acids to reduce G protein-coupled receptor 110 (GPR110) expression in liver of a subject in need thereof for treating non-alcoholic fatty liver disease (NAFLD), conditions and symptoms associated therewith.

12. The pharmaceutical composition of claim 11, wherein the therapeutic nucleic acids are capable of gene silencing of GPR110.

13. The pharmaceutical composition of claim 11, wherein the therapeutic nucleic acids comprise small interfering RNA (siRNA), short-hairpin RNA (shRNA), micro RNA (miRNA), RNA induced silencing complex (RICS), or a complex of clustered regularly interspaced short palindromic repeats (CRISPR) with CRISPR-associated protein 9 (Cas9).

14. The pharmaceutical composition of claim 11, wherein the therapeutic nucleic acids are hepatic GPR110-specific antisense oligonucleotides (ASOs).

15. The pharmaceutical composition of claim 11, wherein the therapeutic nucleic acids are siRNAs each having a nucleotide sequence selected from one of SEQ ID NOs: 35-38.

16. The pharmaceutical composition of claim 11 is administered via intravenous injection, subcutaneous injection, or oral administration.

17. The pharmaceutical composition of claim 11, wherein the conditions or symptoms associated with the NAFLD comprise hepatic steatosis, and chronic noncommunicable diseases (NCDs), non-alcoholic steatohepatitis (NASH) with fibrosis, cirrhosis and hepatocellular carcinoma (HCC), wherein the NCDs comprise type 2 diabetes mellitus (T2DM), cardiovascular disease (CVD), hypertriglyceridemia, atherosclerosis, and chronic kidney disease (CKD).

18. The pharmaceutical composition of claim 11, wherein the subject comprises non-human animals and humans.

19. The pharmaceutical composition of claim 11, further comprising a viral vector.

20. The pharmaceutical composition of claim 19, wherein the viral vector comprises adenoviral, adeno-associated viral, retroviral, or lentiviral vector.


SEQUENCE LISTING (39 sequences)

SEQ ID NO: 1 - protein, 714 aa, Homo sapiens
MQMELKFKNGSIVAGYEVVGSSSASELLSAIEHVAEKAKTALHKLFPLEDGSFRVFGKAQCNDIVFGFGSKDDEYTLPCSSGYRGNITAKCESSGWQVIRETCVLSLLEELNKNFSMIVGNATEAAVSSFVQNLSVIIRQNPSTTVGNLASVVSILSNISSLSLASHFRVSNSTMEDVISIADNILNSASVTNWTVLLREEKYASSRLLETLENISTLVPPTALPLNFSRKFIDWKGIPVNKSQLKRGYSYQIKMCPQNTSIPIRGRVLIGSDQFQRSLPETIISMASLTLGNILPVSKNGNAQVNGPVISTVIQNYSINEVFLFFSKIESNLSQPHCVFWDFSHLQWNDAGCHLVNETQDIVTCQCTHLTSFSILMSPFVPSTIFPVVKWITYVGLGISIGSLILCLIIEALFWKQIKKSQTSHTRRICMVNIALSLLIADVWFIVGATVDTTVNPSGVCTAAVFFTHFFYLSLFFWMLMLGILLAYRIILVFHHMAQHLMMAVGFCLGYGCPLIISVITIAVTQPSNTYKRKDVCWLNWSNGSKPLLAFVVPALAIVAVNFVVVLLVLTKLWRPTVGERLSRDDKATIIRVGKSLLILTPLLGLTWGFGIGTIVDSQNLAWHVIFALLNAFQGFFILCFGILLDSKLRQLLFNKLSALSSWKQTEKQNSSDLSAKPKFSKPFNPLQNKGHYAFSHTGDSSDNIMLTQFVSNE

SEQ ID NO: 2 - protein, 927 aa, Mus musculus
MKFKFAKKQVQRGEKELLDMRIGLLWLVPLFTLTEGTDGFLQQKNDGRRTKEIVSMVEERHPVHEYEVLLQVTYRDPEEKRELKRFLKLLKSPSPSLRGPSKIIRVKATTYCRSRKGFLECACEDSYTWFPPSCLDPQNCCLHTTGPVPSCNCSLRGLRQSINFCERAKVWGTFEIDEKFPEDLWNSSSDVYAHYTVGIENQLKEAYRRVHGFESVRVTQFRKGSIVVGYEVTGSTSPPELLFAIEQEAEKAQEALRRQFPVKYGSFRVFGKAPCNSISFGFGSENDEYTVPCSSGFTGSMTVRCQSSGWQITRESCVLSQLEELKKELRMIAGKITEAGVASLVQNLSTILLQSPSTTVGNLGSVVSLLSNISSLSLANSLTVSNLTLMNVINIADHILDSASITNWTILLQDAKDASSQLLKTLESISSLIPSMALPLNFSGKFIDWKGTPVTQIQSTRGYNYQMEMRQNASLPIRGHVFIEPDQFQKSHPKTIISMASLTFGDILPITQRGNAWVNGPVISTLIQNYSISEIFLNFSKIKGNLTQPRCVFWDFSQLQWSNAGCQLVNETLDTVLCRCSHLTSFSMLMSPFVPSSVVPVVKWITYIGLSISIASLILCLIIESLFWKQTKRSQTSYTRNICLVNIAVSLLIADVWFIIAATVDPSVSPSGVCVAAVFFTHFFYLAVFFWMLVLGILLAYRIILVFHHMALTTMMAIGFCLGYGCPLLISIITLAVTQPSNSYKRNDVCWLNWSDKSKPLLAFVVPALTIVAVNLVVVLLVLRKLWRPAVGERLNQDDKATAIRMGKSLLVLTPLLGLTWGFGIGTMANSHNLAWHVLFALLNAFQGFFIFCFGILLDTKLRQLLSNKLTTLSSWKQTSKRNASDTVTQPKCLRTFNILQHRGMYALSHTGDSSSDITLTQFLSTE

SEQ ID NO: 3 - DNA, 20 bp, synthetic construct
  source 1..20: mol_type = other DNA
ccaagagaagccaaacctcc

SEQ ID NO: 4 - DNA, 20 bp, synthetic construct
  source 1..20: mol_type = other DNA
ttcgataagccagcaggatg

SEQ ID NO: 5 - DNA, 20 bp, synthetic construct
  source 1..20: mol_type = other DNA
ctgacctgaaagccgagaag

SEQ ID NO: 6 - DNA, 20 bp, synthetic construct
  source 1..20: mol_type = other DNA
agaaggtgctaacgaacagg

SEQ ID NO: 7 - DNA, 20 bp, synthetic construct
  source 1..20: mol_type = other DNA
actccactcacggcaaattc

SEQ ID NO: 8 - DNA, 20 bp, synthetic construct
  source 1..20: mol_type = other DNA
tctccatggtggtgaagaca

SEQ ID NO: 9 - DNA, 17 bp, synthetic construct
  source 1..17: mol_type = other DNA
acaggacacctgctctc

SEQ ID NO: 10 - DNA, 23 bp, synthetic construct
  source 1..23: mol_type = other DNA
agtcctgagtccttcatgtcttt

SEQ ID NO: 11 - DNA, 23 bp, synthetic construct
  source 1..23: mol_type = other DNA
ctttggctatgggccttccagtc

SEQ ID NO: 12 - DNA, 24 bp, synthetic construct
  source 1..24: mol_type = other DNA
gcaaggaggacagagtttatcgtg

SEQ ID NO: 13 - DNA, 21 bp, synthetic construct
  source 1..21: mol_type = other DNA
atggacgctgatggcaatacc

SEQ ID NO: 14 - DNA, 19 bp, synthetic construct
  source 1..19: mol_type = other DNA
tccccattcacgtctccca

SEQ ID NO: 15 - DNA, 20 bp, synthetic construct
  source 1..20: mol_type = other DNA
actacgatgacctccccaag

SEQ ID NO: 16 - DNA, 20 bp, synthetic construct
  source 1..20: mol_type = other DNA
catagcaaggccaagttcac

SEQ ID NO: 17 - DNA, 23 bp, synthetic construct
  source 1..23: mol_type = other DNA
gttcctacagatttctagctccc

SEQ ID NO: 18 - DNA, 20 bp, synthetic construct
  source 1..20: mol_type = other DNA
agagtctgccatgatttccg

SEQ ID NO: 19 - DNA, 20 bp, synthetic construct
  source 1..20: mol_type = other DNA
cactcattcctgcccttctc

SEQ ID NO: 20 - DNA, 21 bp, synthetic construct
  source 1..21: mol_type = other DNA
acaatcaccttcagctcactc

SEQ ID NO: 21 - DNA, 21 bp, synthetic construct
  source 1..21: mol_type = other DNA
aaggctatgtgaaggatgtgg

SEQ ID NO: 22 - DNA, 22 bp, synthetic construct
  source 1..22: mol_type = other DNA
ctgtctgaagaggttagggaag

SEQ ID NO: 23 - DNA, 21 bp, synthetic construct
  source 1..21: mol_type = other DNA
ttttatgacctcttccctggc

SEQ ID NO: 24 - DNA, 19 bp, synthetic construct
  source 1..19: mol_type = other DNA
attcgctccaggttccatg

SEQ ID NO: 25 - DNA, 20 bp, synthetic construct
  source 1..20: mol_type = other DNA
tcaagctggttcgagttcag

SEQ ID NO: 26 - DNA, 19 bp, synthetic construct
  source 1..19: mol_type = other DNA
acagagcagttgaggttgg

SEQ ID NO: 27 - DNA, 20 bp, synthetic construct
  source 1..20: mol_type = other DNA
agtgttcagtccattgtccc

SEQ ID NO: 28 - DNA, 19 bp, synthetic construct
  source 1..19: mol_type = other DNA
tgactgccacaacctgaag

SEQ ID NO: 29 - DNA, 19 bp, synthetic construct
  source 1..19: mol_type = other DNA
cctgtgctctaccatgctg

SEQ ID NO: 30 - DNA, 20 bp, synthetic construct
  source 1..20: mol_type = other DNA
attccatatccaggttgccg

SEQ ID NO: 31 - DNA, 20 bp, synthetic construct
  source 1..20: mol_type = other DNA
tgacatggtacatcgaagcc

SEQ ID NO: 32 - DNA, 22 bp, synthetic construct
  source 1..22: mol_type = other DNA
gatttctgacactcccttctgg

SEQ ID NO: 33 - DNA, 19 bp, synthetic construct
  source 1..19: mol_type = other DNA
acgaccaagacactgaagc

SEQ ID NO: 34 - DNA, 20 bp, synthetic construct
  source 1..20: mol_type = other DNA
acccaggatttgaatgaccc

SEQ ID NO: 35 - RNA, 23 nt, synthetic construct
  source 1..23: mol_type = other RNA
aagaactcattgtgaataagaaa

SEQ ID NO: 36 - RNA, 23 nt, synthetic construct
  source 1..23: mol_type = other RNA
ttctgctatatactccaaatatg

SEQ ID NO: 37 - RNA, 23 nt, synthetic construct
  source 1..23: mol_type = other RNA
gagatatctttgcacagataaca

SEQ ID NO: 38 - RNA, 23 nt, synthetic construct
  source 1..23: mol_type = other RNA
aacagcataagaagtgattgagc

SEQ ID NO: 39 - RNA, 5432 nt, Homo sapiens
  source 1..5432: mol_type = mRNA
agtgagcctgtgttcatgccagtgagctgctgtggctcagatactgatactttctttccaaacagcataagaagtgattgagccacaagtatactgaaggaagggctccctcgagttctggtgtgaagagataaatcaccagtcacagactatgcacccgactgctgctgttcagtccagggaaaatgaaagttggagtgctgtggctcatttctttcttcaccttcactgacggccacggtggcttcctggggaaaaatgatggcatcaaaacaaaaaaagaactcattgtgaataagaaaaaacatctaggcccagtcgaagaatatcagctgctgcttcaggtgacctatagagattccaaggagaaaagagatttgagaaattttctgaagctcttgaagcctccattattatggtcacatgggctaattagaattatcagagcaaaggctaccacagactgcaacagcctgaatggagtcctgcagtgtacctgtgaagacagctacacctggtttcctccctcatgccttgatccccagaactgctaccttcacacggctggagcactcccaagctgtgaatgtcatctcaacaacctcagccagagtgtcaatttctgtgagagaacaaagatttggggcactttcaaaattaatgaaaggtttacaaatgaccttttgaattcatcttctgctatatactccaaatatgcaaatggaattgaaattcaacttaaaaaagcatatgaaagaattcaaggttttgagtcggttcaggtcacccaatttcgaaatggaagcatcgttgctgggtatgaagttgttggctccagcagtgcatctgaactgctgtcagccattgaacatgttgccgagaaggctaagacagcccttcacaagctgtttccattagaagacggctctttcagagtgttcggaaaagcccagtgtaatgacattgtctttggatttgggtccaaggatgatgaatataccctgccctgcagcagtggctacaggggaaacatcacagccaagtgtgagtcctctgggtggcaggtcatcagggagacttgtgtgctctctctgcttgaagaactgaacaagaatttcagtatgattgtaggcaatgccactgaggcagctgtgtcatccttcgtgcaaaatctttctgtcatcattcggcaaaacccatcaaccacagtggggaatctggcttcggtggtgtcgattctgagcaatatttcatctctgtcactggccagccatttcagggtgtccaattcaacaatggaggatgtcatcagtatagctgacaatatccttaattcagcctcagtaaccaactggacagtcttactgcgggaagaaaagtatgccagctcacggttactagagacattagaaaacatcagcactctggtgcctccgacagctcttcctctgaatttttctcggaaattcattgactggaaagggattccagtgaacaaaagccaactcaaaaggggttacagctatcagattaaaatgtgtccccaaaatacatctattcccatcagaggccgtgtgttaattgggtcagaccaattccagagatcccttccagaaactattatcagcatggcctcgttgactctggggaacattctacccgtttccaaaaatggaaatgctcaggtcaatggacctgtgatatccacggttattcaaaactattccataaatgaagttttcctatttttttccaagatagagtcaaacctgagccagcctcattgtgtgttttgggatttcagtcatttgcagtggaacgatgcaggctgccacctagtgaatgaaactcaagacatcgtgacgtgccaatgtactcacttgacctccttctccatattgatgtcaccttttgtcccctctacaatcttccccgttgtaaaatggatcacctatgtgggactgggtatctccattggaagtctcattttatgcctgatcatcgaggctttgttttggaagcagattaaaaaaagccaaacctctcacacacgtcgtatttgcatggtgaacatagccctgtccctcttgattgctgatgtctggtttattgttggtgccacagtggacaccacggtgaacccttctggagtctgcacagctgctgtgttctttacacacttcttctacctctctttgttcttctggatgctcatgcttggcatcctgctggcttaccggatcatcctcgtgttccatcacatggcccagcatttgatgatggctgttggattttgcctgggttatgggtgccctctcattatatctgtcattaccattgctgtcacgcaacctagcaatacctacaaaaggaaagatgtgtgttggcttaactggtccaatggaagcaaaccactcctggcttttgttgtccctgcactggctattgtggctgtgaacttcgttgtggtgctgctagttctcacaaagctctggaggccgactgttggggaaagactgagtcgggatgacaaggccaccatcatccgcgtggggaagagcctcctcattctgacccctctgctagggctcacctggggctttggaataggaacaatagtggacagccagaatctggcttggcatgttatttttgctttactcaatgcattccagggattttttatcttatgctttggaatactcttggacagtaagctgcgacaacttctgttcaacaagttgtctgccttaagttcttggaagcaaacagaaaagcaaaactcatcagatttatctgccaaacccaaattctcaaagcctttcaacccactgcaaaacaaaggccattatgcattttctcatactggagattcctccgacaacatcatgctaactcagtttgtctcaaatgaataaggcaaggaatcataaaatcaagaaaaaatttccagaacaacttgacatttagagacaaatgtcaatgaagaaattatgctcagtattcgatcgggttttctgatttaggggtctgggaataaaacaagaatgtctcagtggcttcattactgctcccttttgtcttcaattaaatgaaaagaagatttatttccatgtgatttgattcaaagaaagtgctccataaatgcagaagagtaggttttgttggaaatcgtgtcagttgtaccctgaccataaaatatggtttctattttcataaaacagcattattcacatggcatttccaataatctggattgaaggaagaaaattttatgaaatagctttagataaattaataggccacgttcattttcttgtcaaaaagttactggtggggggatggtgggaaaaagttattagtgcaaatttcctagagaaaaaaccatttctctttcaaattttccagttgaattttatgttcgcttttgcttcttaggttctatcacttaatattgaaagttaatcagaaataaaatgtaaacttctatttcagatagctttgtaaccatttatcagaaagtataataatgtgatatgatatataatgtggtatttttcagtttacaaggcacttccatctggtcctaaaccctgcaaacaaaagtgtcaaggcagacctagtgcagagatgagggcatgggggctcagagaggtaaagtgacttgccaaagattgtgaagccagttaagggaaattggggatttttaggacatttgtctcccagaccatttctacagccaataaaagccttgaaaattacctacatctctagtaaatctttctgaggtcagttacacagttcaccattcccagaattctatgtaagagagaaattaagggagaatttggttgtcagcttccagggaggtaccttaaaacatccttattttctttcataatacaccataggcactttgtggaagttattttcttgtacagccagtgccacttttggatgacatacaaagcagcaggagttgacgctatgtgaagcaacacttctcttccatacttccatcacatttggctcacctacctgagtttcagccgtgaccatagtgcccagaggaacccgagcctttgattcatccttgctgttttgagaggcctagggagtgtctcttagcctcctgacatggaaagccttcatgctatgcaaataaagacagctgggggaacttctagttagtggtggtagattaaataagcatacctgagagaaattcaaagttagacttccacaaggcaccaaggtgcagcacacatgaaatatgaaagtgagaggttcaaaggaaatgactgaggatcagttagatggttctataccccacaaatatgtatgagagtcaacagaattttatgagaaagacttctttgcagaaaactgctctatgagccttctcactctacctacccacaaaagtgagagaaggaggaatgtctgctccttgacctcaaatttagtgagagaagatttctcagatagctcagatagctcagatttctcagaaccacaacaattaggatgcatggctgatggaagtctgattcttgcctagaattgtggccacagctccggtagtagagccctgaatgagttctgtgttgagaatagcctgtatgtctagtgtttgtcagggcaatggatgtgtgtgtttcccatgaaccatagtgagacatgcaagagtgaaggtcagcatggggctcttttaggtcctctctgatagggttgtccaaagaggtggagggaacccagcatcaagaagtgggaggtgaagccagattgtgggatgaggaaggagtcacaaatgaccacctggaatacaaaaaaatctacacaagctaaagatgagaagtcagggctatgagaatttctatgggagttcttgaatgctccctctcttattacacacaattactatccagagaaccctgacaccatgtgaaggcagccctatctcataagacagatttctttttttggggaccttggggaaagggaggccagaaacctccgtgtccaaggtggggtggaaggagagggagaaatcttagcaaaaatgtcaaccagcaagggaagatgtgctagcgcaagtaggcaactcaacacaagactcagttggagagggaagaaaaatttagtactaaattattgtagtttcttcttattgtaggcgactgggagtcataattaccaaactatttaaaatgaccaaaatgttgctcttacctgagtatgttggcatacaactggaatgtatctgatggaagaaaaagcttctcccactgaataaattttatgaggatgatgagagacaaaaaaatgtttgatcagatatcatgtatgtgcatgcttgtctaattcactgattatgcatacattcacctcaaaatgctactaaaacaacaataaaataatgaaaaagtgaa